SEQUENCE LISTING (52 sequences)

SEQ ID NO: 1 - protein, 116 aa, Mus musculus
EVQLQESGGELVRPGTSVKISCKASGYTFTNFWLGWIKERPGHGLEWIGDIYPGGHYTNYNENFKGKATLTADTSSNTAYMQVSSLTSEDSAVYFCFTQYYDMDYWGQGTSVTVSS

SEQ ID NO: 2 - protein, 113 aa, Mus musculus
DVVMTQTPLTLSVTIGQPASISCKSSQSLLYSDGETYLNWLLQRPGQSPKRLIYLVSKLDSGVPDRFTGSGSGTDFTLKISRVEAEDLGVYYCWQGTHFPQTFGGGTKLEIKR

SEQ ID NO: 3 - protein, 115 aa, Mus musculus
EVQLQESGPELVKPGASVKMSCKASGYTFTDYYMNWVKQSHGKSLEWIGDINPYNGDTTYNQKFKGKATLTVDKSSSTAYMQLNSLTSEDSAVYYCASGYEGDYWGQGTTLTVSS

SEQ ID NO: 4 - protein, 112 aa, Mus musculus
  misc_feature (50)..(50): Xaa can be any naturally occurring amino acid
DIVMTQAAFSNPVTLGTSASISCRSSKSLLHSNGITYLYWYLQKPGQSPXLLIYQMSNLASGVPDRFSSSGSGTDFTLRISRVEAEDVGVYYCAQNLELPTFGSGTKLEIKR

SEQ ID NO: 5 - protein, 123 aa, Mus musculus
EVQLQESGPGLVKPGASVKISCKASGYSFTGYYMNWVKQSPEKSLEWIGEINPSTGGTTYNQKFKAKATLTVDKSSSTAYMQLKSLTSEDSAVYYCVRYSHYYGSGSWYFDVWGTGTTVTVSS

SEQ ID NO: 6 - protein, 108 aa, Mus musculus
DIQMTQSPASLSASVGETVTITCRASENIYSNLAWYQQKQGKSPQLLVNAATNLADGVPSRFSGSGSGTQFSLKINSLQPEDFGSYYCQHFYVTPYMFGSGTKLEIKR

SEQ ID NO: 7 - protein, 121 aa, Mus musculus
EVQLQESGPGLVQPSQSLSITCTVSGFSLTSYGVHWVRQSPGKGLEWLGVIWSGGSTDYNAAFISRLSISKDNSKSQVFFKMNSLQADDTAIYYCARNWFDYDNYYSMDYWGQGTSVTVSS

SEQ ID NO: 8 - protein, 108 aa, Mus musculus
DIQMTQTTSSLSASLGDRVTISCRASQDISNYLNWYQQKPDGTVKLLIYYTSRLHSGVPSRFSGSGSGTDFSLTISNQEPEDIATYYCQQYSNLPWTFGGGTKLEIKR

SEQ ID NO: 9 - protein, 118 aa, Mus musculus
EVQLQESGPELKKPGETVKISCKASGYTFTTYGMSWVKQAPGKGLKWMGRINTYSGVPTYADEFKGRFAFSLENSASTAYLQINNLKNEDTATYFCARGDGGHWFANWGQGTLVTVSA

SEQ ID NO: 10 - protein, 108 aa, Mus musculus
DIQMTQSPSSLSASLGGKVTITCKASQDINKNIAWYQHKPGKGPRLLIWYKSALQPGIPSRFSGSGSGRDYSFSISNLEPEDIATYSCLQYDNLPPTFGGGTKLEIKR

SEQ ID NO: 11 - protein, 119 aa, Mus musculus
EVQLQESGPGILQPSQTLSLTCSFSGFSLRTLGMGVGWIRQSSGKGLEWLAHIWWSDENYYNPALKSRLTISKDTSKNQVFLRIANVDTAETATYYCARIAYDGYFDYWGQGTSLTVSS

SEQ ID NO: 12 - protein, 108 aa, Mus musculus
KFPSQLLLLLLFGIPGDRVTITCKASEDIYNRLAWYQQKPGNAPRLLISGATSLETGVPSRFSGSGSGKDYTLSITSLQTEDVATYYCQQYWTTPWTFGGGTKLEIKR

SEQ ID NO: 13 - protein, 115 aa, Mus musculus
EVQLQESGAELVRPGASVTLSCKTSGYTFTDYEMHWVKQTPVHGLEWIGAIDPETGGTAYNQKFKGKATLTADKSSSTAYMELSRLTSEDSAVYYCTGNYATDYWGQGTSVTVSS

SEQ ID NO: 14 - protein, 113 aa, Mus musculus
DIVMSQSPSSLAVSAGEKVTMSCKSSQSLFNSRSRKNYLAWYQLKPGQSPKLLIYWASTRESGVPDRFTGSGSGTDFTLTISSVQAEDLAVYYCKQSNYLWTFGGGTKLEIKR

SEQ ID NO: 15 - protein, 124 aa, Artificial Sequence
  macaque-human chimeric
EVQLVESGGGLVQPGGSLRLSCAASGFTGFTFSDYAFYWVRQAPGKGLEWVGFIRGKAYGGTADYAASVKGRFTISRDNSKNTAYLQMSSLKTEDSAVYYCTSQGVTVATPYHWGQGVLVTVSS

SEQ ID NO: 16 - protein, 115 aa, Artificial Sequence
  macaque-human chimeric
DIVLTQSPLSLPVTPGEPASISCRSSQSLLHSGGKTYLYWYLQKPGQSPQLLIHEVSNRASGVPDRFSGSGSGTDFTLKISRVEAEDVGVYYCMQGIQLPLTFGGGTKVEIKRTV

SEQ ID NO: 17 - protein, 116 aa, Artificial Sequence
  macaque-human chimeric
QVQLQESGPVLVKPSETLSLTCAVSGYSISSAYAWSWIRQPPGKGLEWVGYVGSSSDYNPSLKSRVTISRDTSKNRIFLNLRSLTAADTAVYYCARDRANNSMDVWGRGVLVTVSS

SEQ ID NO: 18 - protein, 116 aa, Artificial Sequence
  macaque-human chimeric
EIVLTQSPDSLGVSLGERVTINCKSSQSLLYSSNNKNYLAWYQQKPGQAPKLLIYWASTRESGVPNRFSGSGSGTDFTLTISGLQAEDVAVYYCQQYYSTPLTFGQGTKVEIKRTV

SEQ ID NO: 19 - protein, 118 aa, Artificial Sequence
  macaque-human chimeric
EVQLVQSGGGLVQPGGSMRLSCEASGLSLSDYFMHWVRQAQGKGLEWIGLIQTKAFTYKTEYPAAVKGRFTISRDDSKNTLYLQMSSLKPEDTALYYCIAVTPDFYYWGQGVLVTVSS

SEQ ID NO: 20 - protein, 110 aa, Artificial Sequence
  macaque-human chimeric
DVVMTQSPSFLSASVGDRVTITCRASQDITINLNWFQHKPGKAPKRLIYVASRLERGVPSRFSGSGSGTEFTLTISSLQPEDFATYYCQQYNNYPLTFGPGTKLDIKRTV

SEQ ID NO: 21 - protein, 113 aa, Artificial Sequence
  macaque-human chimeric
EVQLVQSGAEVKRPGESLTISCKTSGYSFTDSWVAWVRQMPGKGLEWLGSIYPGDSETKYNPSFQGHVTISADKSVTTTYLKWSSLKASDTATYYCVKGSETWGQGVLVTVSS

SEQ ID NO: 22 - protein, 115 aa, Artificial Sequence
  macaque-human chimeric
QLVLTQPASLSASPGASASLTCTFSGGINVAGYNILWYQQKAGSPPRYLLRYKSDSTKDQGSGVPSRFSGSKDASANTGILRISGLQSEDEADYYCAIGHSSGWIFGGGTRLTVL

SEQ ID NO: 23 - protein, 113 aa, Artificial Sequence; identical to SEQ ID NO: 21
  macaque-human chimeric

SEQ ID NO: 24 - protein, 115 aa, Artificial Sequence
  macaque-human chimeric
NFMLTQPASLSASPGASASLTCTFSGGINVAGYNILWYQQKAGSPPRYLLRYKSDSTKDQGSGVPSRFSGSKDASANTGILRISGLQSEDEADYYCAIGHSSGWIFGGGTRLTVL

SEQ ID NO: 25 - protein, 113 aa, Artificial Sequence
  macaque-human chimeric
EVQLVQSGAEVKRPGESLKISCKTSGYTFTSSWISWVRQMPGKGLEWLGSIYPGDSDTRYNPSFQGHVTISADTSIITTHLQWSSLKASDTATYYCVKGSETWGQGVLVTVSS

SEQ ID NO: 26 - protein, 115 aa, Artificial Sequence
  macaque-human chimeric
KPMLTQPTSLSASPGASASLTCTFSGGINVAGYNILWYQQKAGSPPRYLLRYKSDSTKDQGSGVPSRFSGSKDASANTGILRISGLQSEDEADYYCAIGHSSGWIFGGGTRLTVL

SEQ ID NO: 27 - protein, 113 aa, Artificial Sequence
  macaque-human chimeric
EVQLVESGAEVKRPGESLTISCKTSGYSFTGSWISWVRQMPGKGLEWLGSIYPGDSETKYNPSFQGHVTISADKSVTTTYLKWSSLKASDTATYYCVKGSETWGQGVLVTVSS

SEQ ID NO: 28 - protein, 115 aa, Artificial Sequence
  macaque-human chimeric
NFMLTQSASLSASPGASASLTCTFSGGINVAGYHIFWYQQNPGSPPRYLLRYKSDSEKDQGSGVPSRFSGSKDASANTGILRISGLQSEDEADYYCAIGHSSGWIFGGGTRLTVL

SEQ ID NO: 29 - protein, 15 aa, Ebola virus
KVNPEIDTTIGEWAF

SEQ ID NO: 30 - protein, 15 aa, Ebola virus
IDTTIGEWAFWETKK

SEQ ID NO: 31 - protein, 15 aa, Ebola virus
GEWAFWETKKNLTRK

SEQ ID NO: 32 - protein, 15 aa, Ebola virus
WETKKNLTRKIRSEE

SEQ ID NO: 33 - protein, 20 aa, Ebola virus
EIDTTIGEWAFWETKKNLTR

SEQ ID NO: 34 - protein, 20 aa, Ebola virus
NINADIGEWAFWENKKNLSE

SEQ ID NO: 35 - protein, 20 aa, Ebola virus
TVDTGVGEWAFWENKKNFTK

SEQ ID NO: 36 - protein, 20 aa, Ebola virus
KIEPDVGEWAFWETKKNFSC

SEQ ID NO: 37 - protein, 20 aa, Ebola virus
TVDTSMGEWAFWENKKNFTK

SEQ ID NO: 38 - protein, 15 aa, Ebola virus
HYWTTQDEGAAIGLA

SEQ ID NO: 39 - protein, 15 aa, Ebola virus
QDEGAAIGLAWIPYF

SEQ ID NO: 40 - protein, 15 aa, Ebola virus
AIGLAWIPYFGPAAE

SEQ ID NO: 41 - protein, 15 aa, Ebola virus
WIPYFGPAAEGIYTE

SEQ ID NO: 42 - protein, 15 aa, Ebola virus
AAIGLAWIPYFGPAA

SEQ ID NO: 43 - protein, 15 aa, Ebola virus
NAAGIAWIPYFGPGA

SEQ ID NO: 44 - protein, 15 aa, Ebola virus
AAIGLAWIPYFGPAA

SEQ ID NO: 45 - protein, 15 aa, Ebola virus
AAAGLAWIPYFGPAA

SEQ ID NO: 46 - protein, 15 aa, Ebola virus
AAIGLAWIPYFGPAA

SEQ ID NO: 47 - protein, 15 aa, Marburg virus
LAAGLSWIPFFGPGI

SEQ ID NO: 48 - protein, 15 aa, Marburg virus
LAAGLSWIPFFGPGI

SEQ ID NO: 49 - protein, 15 aa, Marburg virus
LAAGLSWIPFFGPGI

SEQ ID NO: 50 - protein, 15 aa, Marburg virus
LAAGLSWIPFFGPGI

SEQ ID NO: 51 - protein, 15 aa, Marburg virus
LAAGLSWIPFFGPGI

SEQ ID NO: 52 - protein, 15 aa, Ebola virus
AAIGLAWIPYFGPAA

(19) United States
(12) Patent Application Publication
(10) Pub. No.: US 2018/0237502 A1
(43) Pub. Date: Aug. 23, 2018

(54) PAN-EBOLA AND PAN-FILOVIRUS PROTECTIVE EPITOPES, ANTIBODIES, AND ANTIBODY COCKTAILS

(71) Applicant: Integrated BioTherapeutics, Inc., Rockville, MD (US)

(72) Inventors: Mohammad Javad Aman, Rockville, MD (US); Frederick Wayne Holtsberg, Taneytown, MD (US); Sven G. Enterlein, Harpers Ferry, WV (US); Katie A. Howell, Baltimore, MD (US)

(73) Assignees: Integrated BioTherapeutics, Inc., Rockville, MD (US); Integrated BioTherapeutics, Inc., Rockville, MD (US)

(21) Appl. No.: 15/556,898

(22) PCT Filed: Mar. 11, 2016
(86) PCT No.: PCT/US16/22141

§ 371 Date: Sep. 8, 2017

Related U.S. Application Data
(60) Provisional application No. 62/131,563, filed on Mar. 11, 2015.

(51) Int. Cl. C07K 16/10; A61P 31/14

(52) U.S. Cl. CPC C07K 16/10; A61P 31/14; C07K 2317/92; C07K 2317/76; A61K 2039/507; C07K 2317/31; C07K 2317/56; C07K 2317/565; C07K 2317/24

(57) ABSTRACT

This disclosure provides a method for preventing, treating, or managing a filovirus infection in a subject, where the method includes administering to a subject in need thereof an effective amount of at least one binding molecule that includes a binding domain that specifically binds to an orthologous filovirus glycoprotein epitope, wherein the binding domain specifically binds to the epitope on two or more filovirus species or strains.

### BACKGROUND

Filoviruses, ebolavirus and marburgvirus, cause severe hemorrhagic fevers in humans, with mortality rates reaching 88% (Feldmann, et al., 2003, Nat Rev Immunol, 3 (8):677-685) as well as epizootic diseases in nonhuman primates and probably other mammals. Due to weaponization of marburgvirus by the USSR, the high fatality rates, and the potential for aerosol transmission, filoviruses have been classified as Category A NIAID Priority Pathogens. There are currently no vaccines or therapeutics against filoviruses. The main filovirus species causing outbreaks in humans are ebolaviruses Zaire (EBOV) and Sudan (SUDV), as well as the Lake Victoria Marburg virus (MARV). Filoviruses are enveloped, single-stranded, negative sense RNA filamentous viruses and encode seven proteins, of which the spike glycoprotein (GP) is considered the main protective antigen. EBOV and MARV GP is proteolytically cleaved by furin protease into two subunits linked by a disulfide linkage: GP1 (˜140 kDa) and GP2 (˜38 kDa) (Manicassamy, et al., 2005, J Virol, 79 (8):4793-4805). Three GP1-GP2 units form the trimeric GP envelope spike (˜550 kDa) on the viral surface (Feldmann, et al., 1993, Arch Virol Suppl, 7:81-100; Feldmann, et al., 1991, Virology, 182 (1):353-356; Geisbert and Jahrling, 1995, Virus Res, 39 (2-3):129-150; Kiley, et al., 1988a, J Gen Virol, 69 (Pt 8):1957-1967). GP1 mediates cellular attachment (Kiley, et al., 1988b, J Gen Virol, 69 (Pt 8):1957-1967; Kuhn, et al., 2006, J Biol Chem, 281 (23):15951-15958), and contains a mucin-like domain (MLD) which is heavily glycosylated and variable and has little or no predicted secondary structure (Sanchez, et al., 1998, J Virol, 72 (8):6442-6447).

It is well established that the filovirus GPs represent the primary protective antigens (Feldmann, et al., 2003, Nat Rev Immunol, 3 (8):677-685; Feldmann, et al., 2005, Curr Opin Investig Drugs, 6 (8):823-830; Geisbert, et al., 2010, Rev Med Virol, 20(6):344-57). GP consists of a receptor binding GP1 subunit connected with the GP2 fusion domain via a disulfide link. We have previously identified a specific region of the MARV and EBOV GP1 consisting of ˜150 amino acids (Kuhn, et al., 2006, J Biol Chem, 281 (23):15951-15958) that binds filovirus receptor-positive cells, but not receptor-negative cells, more efficiently than GP₁, and competes with the entry of the respective viruses (Kuhn, et al., 2006, J Biol Chem, 281 (23):15951-15958). These properties arc similar to regions defined for SARS coronavirus and Machupo arenavirus (Li, et al., 2003, Nature, 426 (6965):450-454; Radoshitzky, et al., 2007, Nature, 446 (7131):92-96; Wong, et al., 2004, J Biol Chem, 279 (5):3197-3201). This region of GP is referred to here as receptor binding region (RBR) and is part of a larger domain that excludes the highly variable, glycosylated, and bulky mucin-like domain (MLD). The RBR shows the highest level of homology between Filovirus glycoproteins (Kuhn, et al., 2006, J Biol Chem, 281 (23):15951-15958). Therefore, the RBR represents a potential target for pan-filovirus antibodies.

The crystal structure of a trimeric, pre-fusion conformation of EBOV GP (lacking MLD) in complex with an EBOV-specific neutralizing antibody, KZ52, was solved at 3.4 Å (Lee, et al., 2008, Nature, 454 (7200:177-182). In this structure, three GP1 subunits assemble to form a chalice, cradled in a pedestal of the GP2 fusion subunits, while the MLD restricts access to the conserved RBR, sequestered in the GP chalice bowl. Ebola and Marburg GPs are cleaved by cathepsin proteases as an essential step in entry reducing GP1 to an ˜18 kDa product (Chandran, et al., 2005, Science, 308 (5728):1643-1645; Kaletsky, et al., 2007, J Virol, 81 (24):13378-13384; Schornberg, et al., 2006, J Virol, 80 (8):4174-4178). The structures suggest that the most likely site of cathepsin cleavage is the flexible β13-β14 loop of GP1 and illustrate how cleavage there would release the heavily glycosylated regions from GP, leaving just the core of GP1, encircled by GP2, with the RBR now well exposed. Cathepsin cleavage enhances attachment, presumably as a result of better exposing the RBR for interaction with cell surface factors trafficked with the virus into the endosome (Dube, et al., 2009, J Virol, 83:2883-2891). On the surface of the authentic virus, the MLD probably dominates host-interaction surfaces of filovirus GP, and indeed, antibodies against the MLD have been frequently identified.

Role of Antibodies in Protection Against Filovirus Hemorrhagic Fever:

While both T and B cell responses are reported to play a role in protective immune responses to filoviruses (Warfield, et al., 2005, J Immunol, 175 (2):1184-1191), a series of recent reports indicate that antibody alone can provide significant protection. Dye et al. showed that purified convalescent IgG from macaques can protect NHPs against challenge with MARV and EBOV when administered as late as 48 h post exposure (Dye, et al., 2012, Proc Natl Acad Sci USA, 109(13):5034-9). Olinger et al. reported significant protection from EBOV challenge in NHPs treated with a cocktail of three monoclonal antibodies (mAbs) to GP administered 24 h and 48 h post exposure (Olinger, et al., 2012, Proc Natl Acad Sci USA, 109 (44):18030-18035). Similar results were also reported in two other studies (Qiu, et al., 2013, Sci Transl Med, 5 (207):207ra143; Qiu, et al., 2013, J Virol, 87 (13):7754-7757). A recent study shows that a combination of three monoclonal antibodies called ZMapp can protect monkeys when administered five days after exposure to EBOV, at a time when the disease is fully manifest and the viremia is at its peak (Qiu, et al., 2014, Nature, Epub ahead of print doi: 10.1038/nature13777). Collectively these data demonstrate the ability of the humoral response to control filovirus infection. However, the protective monoclonal antibodies reported to-date are species-specific and do not protect against heterologous members of the filoviridae family.

### SUMMARY

This disclosure provides a method for preventing, treating, or managing a filovirus infection in a subject, where the method includes administering to a subject in need thereof an effective amount of at least one binding molecule that includes a binding domain that specifically binds to an orthologous filovirus glycoprotein epitope, wherein the binding domain specifically binds to the epitope on two or more filovirus species or strains. In certain aspects, the binding domain can bind to the orthologous epitope as expressed in two or more, three or more, four or more, or five or more of Marburg virus (MARV), Ravn virus (RAVV), Tai Forest virus (TAFV), Reston virus (RESTV), Sudan virus (SUDV) Ebola virus (EBOV), Bundibugyo virus (BDBV), or any strain thereof.

In certain aspects of the provided method, the ability of the binding molecule to prevent, treat, or manage a filovirus infection can be measured in a model system that includes administering the binding molecule to a group of mice and challenging mice with a mouse-adapted (MA) filovirus before, at the same time as, or after administering the binding molecule to the mice. In certain aspects, the MA-filovirus is a MA ebolavirus (MA-EBOV), a MA-marburgvirus (MA-MARV), or a combination thereof. In the model, the mice can be monitored for effects including, but not limited to increased survival time, decreased weight loss, or a combination thereof as compared to control mice.

According to the provided method, at least one binding molecule to be administered can bind to the same orthologous epitope as the murine monoclonal antibody m2D8, m21D10, m16G8, m17C6, m8C4, m4B8, or m21B2, the macaque monoclonal antibody FVM02P, FVM03, FVM04, FVM09, FVM11, FVM13, or FVM20, or any combination thereof. For example, at least one binding molecule to be administered can bind to the same orthologous epitope as an antibody or antigen-binding fragment thereof that includes a heavy chain variable region (VH) and light chain variable region (VL) that includes the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively, or any combination thereof.

In certain aspects, the binding molecule is an antibody or antigen-binding fragment thereof. For example, according to the provided method the subject can be administered an effective amount of an antibody or antigen-binding fragment thereof that includes VL-CDR1, VL-CDR2, VL-CDR3, VH-CDR1, VH-CDR2, and VH-CDR3 amino acid sequences identical or identical except for four, three, two, or one single amino acid substitutions, deletions, or insertions to the CDRs contained in the VH and VL sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively. In other examples, the subject can be administered an effective amount of an antibody or antigen-binding fragment thereof that includes VH and VL amino acid sequences at least 85%, 90%, 95%, or 100% identical to reference amino acid sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

In certain aspects of the provided method, the subject is administered an effective amount of a combination of at least two, at least three, at least four, or more binding molecules, e.g., antibodies or fragments thereof as described above, where each binding molecule can include a binding domain that specifically binds to an orthologous filovirus glycoprotein epitope, where the binding domain specifically binds to the epitope on two or more filovirus species or strains. In certain aspects, the combination of binding molecules can prevent, treat, or manage filovirus infection in the subject with a potency that is greater than the additive potency of the binding molecules when administered individually. In certain aspects, the potency levels of the ability of the combined binding molecules to prevent, treat, or manage a filovirus infection can be measured in a model that includes administering the binding molecules to a group of mice and challenging mice with a MA-filovirus before, at the same time as, or after administering the binding molecule to the mice. In certain aspects the MA-filovirus can be MA-EBOV, MA-marburgvirus MA-MARV, or a combination thereof. In the model, the mice can be monitored for effects including, but not limited to increased survival time, decreased weight loss, or a combination thereof as compared to control mice.

In certain aspects, the combination at least two, at least three, at least four, or more antibodies or antigen-binding fragments thereof can include monoclonal antibodies m8C4 and FVM09, monoclonal antibodies m8C4 and m16G8, or monoclonal antibodies FVM09 and FVM02P.

In certain aspects of the provided method, the orthologous epitope or epitopes can be in the GP-1 subunit of the viral glycoprotein and/or in the glycan cap region of GP-1. For example, the epitope can be contained within the consensus sequence IN/M-G-E-W-A-F-W-E-N/T-K-K-N-F/L-T/S-K/Q/E. For example, the epitope can be contained in or can include the amino acid sequence G-E-W-A-F. Epitopes contained within, consisting of, or comprising any of these sequences are also provided.

In certain aspects of the provided method, the orthologous epitope or epitopes can be situated at the tip of the fusion loop of the filovirus glycoprotein. For example, the epitope can be contained within the consensus sequence A-I/A-G-L/I-A-W-I-P-Y-F-G-P-A/G-A in BDBV, TAFV, RESTV, SUDV, or EBOV, or within the consensus sequence A-A-G-L-S-W-I-P-F-F-G-P-G-I in MARV or RAVN. For example the epitope can be contained in and or can include the amino acid sequence A-I-G-L-A-W-I-P-Y-F, A-A-G-L-A-W-I-P-Y-F, A-A-G-I-A-W-I-P-Y-F, and/or A-A-G-L-S-W-I-P-F-F. Epitopes contained within, consisting of, or comprising any of these sequences are also provided.

In certain aspects of the provided method, the subject is administered an effective amount of a combination of at least one, at least two, at least three, at least four, or more antibodies where the antibodies can be, e.g., murine antibodies, non-human primate (NHP) antibodies, humanized antibodies, chimeric antibodies, fragments thereof, or any combination thereof. In certain aspects the at least one, at least two, at least three, at least four, or more antibodies are monoclonal antibodies, components of a polyclonal antibody mixture, recombinant antibodies, multispecific antibodies, fragments thereof, or any combination thereof. In certain aspects, the at least one, at least two, at least three, at least four, or more antibodies are bispecific antibodies or fragments thereof. In certain aspects the at least one, at least two, at least three, at least four, or more bispecific antibodies can recognize two different surface exposed and accessible epitopes on a filovirus virion particle. For example, the two different epitopes can be situated in the glycan cap of the filovirus glycoprotein, the tip of the fusion loop of the filovirus glycoprotein, the mucin-like domain, the GP2 fusion domain, or any combination thereof.

In certain aspects of the provided method, the at least one, at least two, at least three, at least four, or more antibodies can include a heavy chain constant region or fragment thereof, e.g., a murine constant region or fragment thereof, a macaque constant region or fragment thereof, or a human constant region or fragment thereof. In certain aspects the heavy chain constant region or fragment thereof can be an IgM, IgG, IgA, IgE, IgD, or IgY constant region or fragment thereof.

In certain aspects of the provided method, the at least one, at least two, at least three, at least four, or more antibodies can further include a light chain constant region or fragment thereof, e.g., a murine constant region or fragment thereof, a macaque constant region or fragment thereof, or a human constant region or fragment thereof.

In certain aspects of the provided method, the at least one, at least two, at least three, at least four, or more antibodies can be an antibody fragment, e.g., an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects of the provided method, the at least one, at least two, at least three, at least four, or more antibodies are conjugated to an antiviral agent, a protein, a lipid, a detectable label, a polymer, or any combination thereof.

In certain aspects of the provided method, the filovirus infection is hemorrhagic fever. In certain aspects of the provided method, the subject is a nonhuman primate or a human.

The disclosure further provides an isolated, non-naturally-occurring peptide consisting of no more than 25, 50, 75, or 100 amino acids that includes the amino acid sequence G-E-W-A-F. In certain aspects the isolated, non-naturally-occurring peptide can include the amino acid sequence X1-G-E-W-A-F-W-E-X2-K-K-N-X3-X4-X5, wherein X1 is I, V, or M, X2 is N or T, X3 is F or L, X4 is T or S, and X5 is K, Q, or E. The disclosure further provides an isolated, non-naturally-occurring peptide consisting of no more than 25, 50, 75, or 100 amino acids that includes the amino acid sequence A-I-G-L-A-W-I-P-Y-F, A-A-G-L-A-W-I-P-Y-F, A-A-G-I-A-W-I-P-Y-F, or A-A-G-L-S-W-I-P-F-F. In certain aspects the isolated, non-naturally-occurring peptide can include the amino acid sequence the amino acid sequence A-X1-G-X2-A-W-I-P-Y-F-G-P-X3-A, wherein X1 is I or A, X2 is L or I, and X3 is A or G, or the amino acid sequence A-A-G-L-S-W-I-P-F-F-G-P-G-I. In certain aspects, the isolated, non-naturally-occurring peptide provided by the disclosure can be is attached to a heterologous moiety, for example, an antimicrobial agent, a therapeutic agent, a prodrug, a peptide, a linker, a scaffold, a protein, an enzyme, a lipid, a biological response modifier, a pharmaceutical agent, a lymphokine, a heterologous antibody or fragment thereof, a detectable label, a polyethylene glycol (PEG), and any combination thereof.

The disclosure further provides an immunogenic composition that includes one or more of the isolated, non-naturally-occurring peptides provided herein. The disclosure further includes a method of producing an antibody or antigen-binding fragment thereof that specifically binds to an orthologous filovirus glycoprotein epitope, comprising administering the immunogenic composition as provided herein to an animal, and recovering the antibody or fragment thereof.

### BRIEF DESCRIPTION OF THE DRAWINGS/FIGURES

FIG. 1: Competition of Pools of overlapping EBOV GP peptides with binding of FVM09 (A) and FVM11 (B) to EBOV GP immobilized on ELISA plate; pool 12 competed with binding. C) Individual peptides in pool 12 were tested for competition with binding of FVM11 to EBOV GP. Peptides Z57 and Z58 competed with binding.

FIG. 2: A) Sequences of overlapping peptides spanning the epitope for FVM09 and FVM11. The core of the epitope is shown in a gray-shaded box. B) sequence identity between the different ebolavirus species in the region spanning the epitope for FVM09 and FVM11. C) position of the FVM09/11 epitope within the disordered loop connecting 1317-1318 strands.

FIG. 3: Competition of Pools of overlapping EBOV GP peptides with binding of FVM02P (A) to EBOV GP immobilized on ELISA plate; pool 21 and 22 competed with binding. (B) individual peptides were tested for competition with binding of FVM02P to EBOV GP. Peptides Z105 and Z106 competed with binding.

FIG. 4: A) Sequences of overlapping peptides spanning the epitope for FVM02P. The core of the epitope is shown in a gray-shaded box. B) Sequence identity between the different ebolavirus species and marburgvirus strains in the region spanning the epitope for FVM02P. C) the position of FVM02P epitope within the fusion loop of EBOV GP shown in the trimeric crystal structure. The epitope (shown in yellow and encircled) is at the tip of the fusion loop (red+yellow) and makes contacts with both GP1 (light gray) and GP2 (orange) of the adjacent GP molecule.

FIG. 5: A) Protection against EBOV challenge by pan-Ebola antibodies FVM04 and FVM20 shown as % survival in groups of 5 mice. B) Average % weight loss after challenge in mice treated with FVM04 and FVM20 in comparison with the control mice.

FIG. 6: A) Study design for efficacy of m8C4 in SUDV infection model. B) Protection against SUDV challenge by Pan-Ebola m8C4 shown as % survival in groups of 10 mice. 16F6, a SUDV specific (non-cross reactive) is shown as positive control. C) Average % weight loss after challenge in mice treated with m8C4 in comparison with the control mice.

FIG. 7: A) Synergistic protection against EBOV challenge by combination of two glycan cap binders m8C4 and FVM09. B) Average % weight loss after challenge in mice treated with m8C4 and FVM09 in comparison with the control mice.

FIG. 8: A) Synergistic protection against EBOV challenge by combination of the glycan cap binders m8C4 and m16G8, a Pan-Ebola antibody binding to an unknown conformational epitope. B) Average % weight loss after challenge in mice treated with m8C4 and m16G8 in comparison with the control mice.

FIG. 9: A) Synergistic protection against EBOV challenge by combination of the glycan cap binders FVM09 and FVM02P, a Pan-Filovirus antibody binding to a linear epitope within the fusion loop of all filovirus glycoproteins. B) Average % weight loss after challenge in mice treated with FVM09 and FVM02P in comparison with the control mice.

### DETAILED DESCRIPTION

The term “a” or “an” entity refers to one or more of that entity; for example, “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or could be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or could be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, deletions or additions. Derivatives are polypeptides that have been altered so as to exhibit additional features not found on the native polypeptide. Examples include fusion proteins. Variant polypeptides can also be referred to herein as “polypeptide analogs.” As used herein a “derivative” refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

Disclosed herein are certain binding molecules, or antigen-binding fragments, variants, or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

As used herein, the term “binding molecule” refers in its broadest sense to a molecule that specifically binds an antigenic determinant. As described further herein, a binding molecule can comprise one of more “binding domains.” As used herein, a “binding domain” or “antigen binding domain” is a two- or three-dimensional structure, e.g., a polypeptide structure that cans specifically bind a given antigenic determinant, or epitope. One example of a binding domain is the region formed by the heavy and light chain variable regions of an antibody or fragment thereof. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen-binding site present at the end of each arm of the Y. More specifically, the antigen-binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acids when compared against each other. Nevertheless, application of either definition to refer to a CDR of an antibody or variants thereof is intended to be within the scope of the term as defined and used herein. The appropriate amino acids that encompass the CDRs as defined by each of the above-cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of CDR definitions in Table 1 is according to the numbering conventions set forth by Kabat et al. (see below).

Immunoglobulin variable domains can also be analyzed using the IMGT information system (www://imgt.cines.fr/) (IMGT®/V-Quest) to identify variable region segments, including CDRs. See, e.g., Brochet, X. et al., Nucl. Acids Res. 36:W503-508 (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

This disclosure provides cross-reactive binding molecules that bind derived from non-human primate (NHP) antibody binding domains. A “Murine- and/or NHP-derived” binding molecule, e.g., an antibody or antigen-binding fragment thereof, can include any portion of an antibody binding domain, e.g., a single CDR, three CDRs, six CDRs, a VH, a VL, or any combination thereof derived from a mouse and/or a non-human primate (NHP) antibody, e.g., an antibody produced by B cells of a mouse and/or NHP, e.g., a macaque e.g., a rhesus macaque (Macaca mulatta), or a cynomolgus macaque (Macaca fascicularis).

A Murine- and/or NHP-derived binding molecule, e.g., an antibody or fragment, variant, or derivative thereof disclosed herein can be said to bind a target antigen, e.g., a filovirus glycoprotein subunit disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. A Murine- and/or NHP-derived binding molecule as disclosed herein can be said to bind a target antigen, e.g., a filovirus glycoprotein subunit, with an off rate (k(off)) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

A Murine- and/or NHP-derived binding molecule, e.g., an antibody or antigen-binding fragment, variant, or derivative disclosed herein can be said to bind a target antigen, e.g., a filovirus glycoprotein subunit with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. A Murine- and/or NHP-derived binding molecule as disclosed herein can be said to bind a target antigen, e.g., a filovirus glycoprotein subunit with an on rate (k(on)) greater than or equal to 10⁵ M⁻¹ sec, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A Murine- and/or NHP-derived binding molecule, e.g., an antibody or fragment, variant, or derivative thereof can be said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A Murine- and/or NHP-derived binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a Murine- and/or NHP-derived binding molecule, e.g., an antibody or fragment, variant, or derivative thereof, specific for one antigen, to react with a second antigen; a measure of relatedness between two different antigenic substances. Thus, a binding molecule is cross-reactive if it binds to an epitope other than the one that induced its formation, e.g., various different filovirus receptor binding regions. The cross-reactive epitope contains many of the same complementary structural features as the inducing epitope, and in some cases, can actually fit better than the original.

A Murine- and/or NHP-derived binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻²M, 10⁻²M, 5×10⁻³ M, 10⁻³ M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹ M, 10⁻⁹ M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³ M, 5×10⁻¹⁴ M, 10⁻¹⁴ M, 5×10⁻¹⁵ M, or 10⁻¹⁵ M.

Antibody fragments including single-chain antibodies can comprise the variable region(s) alone or in combination with the entirety or a portion of the following: hinge region, CH1, CH2, and CH3 domains. Also included are antigen-binding fragments that comprise any combination of variable region(s) with a hinge region, CH1, CH2, and CH3 domains. Binding molecules, e.g., antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain, a Murine- and/or NHP-derived binding molecule, e.g., an antibody comprising a heavy chain portion comprises at least one of: a CH1 domain, a hinge (e.g., upper, middle, and/or lower hinge region) domain, a CH2 domain, a CH3 domain, or a variant or fragment thereof. For example, a Murine- and/or NHP-derived binding molecule, e.g., an antibody or fragment, variant, or derivative thereof can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a Murine- and/or NHP-derived binding molecule, e.g., an antibody or fragment, variant, or derivative thereof comprises a polypeptide chain comprising a CH3 domain. Further, a Murine- and/or NHP-derived binding molecule for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a Murine- and/or NHP-derived binding molecule, e.g., an antibody as disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion comprises at least one of a VL or CL domain.

Murine- and/or NHP-derived binding molecules, e.g., antibodies or antigen-binding fragments, variants, or derivatives thereof disclosed herein can be described or specified in terms of the epitope(s) or portion(s) of an antigen, e.g., a target a filovirus glycoprotein subunit that they recognize or specifically bind. The portion of a target antigen that specifically interacts with the antigen-binding domain of an antibody is an “epitope,” or an “antigenic determinant.” A target antigen, e.g., a filovirus glycoprotein subunit can comprise a single epitope, but typically comprises at least two epitopes, and can include any number of epitopes, depending on the size, conformation, and type of antigen. As used herein, an “orthologous epitope” refers to versions of an epitope found in related organisms, e.g., different filovirus species or strains. Orthologous epitopes can be similar in structure, but can vary in one or more amino acids.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about amino acid 244 to amino acid 360 of an antibody using conventional numbering schemes (amino acids 244 to 360, Kabat numbering system; and amino acids 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 amino acids.

As used herein, the term “hinge region” includes the portion of a heavy chain molecule that joins the CH1 domain to the CH2 domain. This hinge region comprises approximately 25 amino acids and is flexible, thus allowing the two N-terminal antigen-binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more CDRs from an antibody of known specificity and, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” In some instances, not all of the CDRs are replaced with the complete CDRs from the donor variable region to transfer the antigen binding capacity of one variable domain to another, instead, minimal amino acids that maintain the activity of the target-binding site are transferred. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that could be at any time, determined or interpreted by a judge or an administrative or judicial body to be, “naturally-occurring.”

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picomaviruses (particularly an internal ribosome entry site, or IRES, also referred to as a CITE sequence).

In other embodiments, a polynucleotide can be RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide as disclosed herein, e.g., a polynucleotide encoding a polypeptide subunit provided herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” or “treatment of” (e.g., in the phrase “treating a subject”) refers to reducing the potential for disease pathology, reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals, including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective, and that contains no additional components that are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

Certain therapies can provide “synergy” and prove “synergistic”, i.e., an effect can be achieved when the active ingredients used together that is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

Pan-Filovirus Binding Molecules and Treatment Methods

This disclosure provides methods for treating, preventing, ameliorating or suppressing symptoms of filovirus infection, e.g., EBOV, SUDV, or MARV infection comprising administering to a subject in need thereof a pan-filovirus binding molecule, e.g., a cross-reactive anti-filovirus antibody or antigen-binding fragment thereof containing at least a portion of a mouse or non-human primate antibody, e.g., an antibody provided herein or in US provisional Application Nos. 62/019,668, filed, Jul. 1, 2014, and 62/069,664, filed Oct. 28, 2014, both of which are incorporated herein by reference in their entireties, e.g., at least one CDR, at least three CDRs, at least six CDRs, at least a VH, at least a VL, or at least a VH and a VL derived from a mouse, or a non-human primate, e.g., a macaque, e.g., a rhesus macaque (Macaca mulatta). In certain aspects the method includes administering two, three, four, five or more of such binding molecules to, e.g., improve efficacy, reduce the number of treatments, to allow efficacy when administered at a later time from the inception of infection in the subject, and/or to allow dose sparing. In certain aspects administration of two or more binding molecules as a combination therapy can result in synergistic efficacy, e.g., efficacy that is more potent than would be expected based on the efficacy of the binding molecules administered individually. Pan-filovirus binding molecules, e.g., Murine- and/or NHP-derived binding molecules as provided herein, as well as combinations thereof can be useful for treatment of a filovirus infection without it being necessary to know the exact filovirus species or strain. More specifically, the disclosure provides methods of using an isolated Murine- and/or NHP-derived binding molecule or antigen-binding fragment thereof comprising a binding domain that specifically binds to an orthologous filovirus glycoprotein epitope, wherein the binding domain specifically binds to the epitope on two, three, four, five, or more filovirus species or strains. In certain aspects, the disclosure further provides such orthologous filovirus glycoprotein epitopes. In certain aspects the pan-filovirus binding molecule, e.g., a murine- and/or NHP-derived binding molecule as provided herein can be a cross-reactive antibody or antigen-binding fragment thereof. In certain aspects the binding molecule can be a bispecific antibody that can facilitate targeting of the binding molecule to the endosomal region of a filovirus-infected cell, e.g., through a second binding domain. See, e.g., Provisional Patent Appl. Ser. No. 62/019,668, filed, Jul. 1, 2014, which is incorporated herein by reference in its entirety.

In certain aspects, the binding domain of a pan-filovirus binding molecule, e.g., a murine- and/or NHP-derived binding molecule, e.g., a cross-reactive anti-filovirus antibody or antigen-binding fragment thereof for use in the methods provided herein can specifically bind to a filovirus orthologous epitope as expressed in one or more, two or more, three or more, four or more, or five or more filovirus species or strains thereof, including, Marburg virus (MARV), Ravn virus (RAVV), Tai Forest virus (TAFV), Reston virus (RESTV), Sudan virus (SUDV), Ebola virus (EBOV), and Bundibugyo virus (BDBV). For example, the binding domain of a pan-filovirus binding molecule, e.g., a murine- and/or NHP-derived binding molecule, e.g., a cross-reactive anti-filovirus antibody or antigen-binding fragment thereof for use in the methods provided herein can bind to an orthologous filovirus epitope as expressed in one or more, two or more, or three of EBOV, SUDV, MARV, RESTV, and BDBV. In certain aspects, the binding domain of a pan-filovirus binding molecule, e.g., a murine- and/or NHP-derived binding molecule, e.g., a cross-reactive anti-filovirus antibody or antigen-binding fragment thereof for use in the methods provided herein can bind to an orthologous filovirus epitope as expressed in MARV. In certain aspects, the binding domain of a pan-filovirus binding molecule, e.g., a murine- and/or NHP-derived binding molecule, e.g., a cross-reactive anti-filovirus antibody or antigen-binding fragment thereof for use in the methods provided herein can bind to an orthologous filovirus epitope as expressed in EBOV and SUDV. Any filovirus epitope which has similarities across filovirus species can be a target of the binding domain of a pan-filovirus binding molecule, e.g., a murine- and/or NHP-derived binding molecule as provided herein. In certain aspects, the orthologous epitope can be in the GP-1 subunit of the filovirus glycoprotein, e.g., in the receptor-binding region (RBR) of GP-1 subunit of the viral glycoprotein.

In certain aspects the orthologous epitope is situated in the glycan cap region of GP-1. For example the orthologous epitope can, in some aspects, be contained within the consensus sequence I/V/M-G-E-W-A-F-W-E-N/T-K-K-N-F/L-T/S-K/Q/E. In certain aspects, the orthologous epitope is contained within or comprises the amino acid sequence G-E-W-A-F. The disclosure further provides an isolated, non-naturally-occurring peptide consisting of no more than 15 amino acids, no more than 20 amino acids, no more than 25 amino acids, no more than 30 amino acids, no more than 35 amino acids, no more than 40 amino acids, no more than 45 amino acids, no more than 50 amino acids, no more than 55 amino acids, no more than 60 amino acids, no more than 65 amino acids, no more than 70 amino acids, no more than 75 amino acids, no more than 80 amino acids, no more than 85 amino acids, no more than 90 amino acids, no more than 95 amino acids, or no more than 100 amino acids, comprising the amino acid sequence G-E-W-A-F. In certain aspects the isolated, non-naturally-occurring peptide comprises the amino acid sequence X1-G-E-W-A-F-W-E-X2-K-K-N-X3-X4-X5, wherein X1 is 1, V, or M, X2 is N or T, X3 is F or L, X4 is T or S, and X5 is K, Q, or E.

In certain aspects, the orthologous epitope is situated at the tip of the fusion loop of the filovirus glycoprotein. For example the orthologous epitope can, in some aspects, be contained within the consensus sequence A-I/A-G-L/I-A-W-I-P-Y-F-G-P-A/G-A in BDBV, TAFV, RESTV, SUDV, or EBOV, or within the sequence A-A-G-L-S-W-I-P-F-F-G-P-G-I in MARV or RAVN. In certain aspects, the epitope is contained within or comprises the amino acid sequence A-I-G-L-A-W-I-P-Y-F, A-A-G-L-A-W-I-P-Y-F, A-A-G-I-A-W-I-P-Y-F, or A-A-G-L-S-W-I-P-F-F. The disclosure further provides an isolated, non-naturally-occurring peptide consisting of no more than 15 amino acids, no more than 20 amino acids, no more than 25 amino acids, no more than 30 amino acids, no more than 35 amino acids, no more than 40 amino acids, no more than 45 amino acids, no more than 50 amino acids, no more than 55 amino acids, no more than 60 amino acids, no more than 65 amino acids, no more than 70 amino acids, no more than 75 amino acids, no more than 80 amino acids, no more than 85 amino acids, no more than 90 amino acids, no more than 95 amino acids, or no more than 100 amino acids, comprising the amino acid sequence G-E-W-A-F. in certain aspects the isolated, non-naturally-occurring peptide comprises the amino acid sequence A-I-G-L-A-W-I-P-Y-F, A-A-G-L-A-W-I-P-Y-F, A-A-G-I-A-W-I-P-Y-F, or A-A-G-L-S-W-I-P-F-F. In certain aspects the isolated, non-naturally-occurring peptide comprises the amino acid sequence A-X1-G-X2-A-W-I-P-Y-F-G-P-X3-A, wherein X1 is I or A, X2 is L or I, and X3 is A or G, or the amino acid sequence A-A-G-L-S-W-I-P-F-F-G-P-G-I.

In certain aspects, an isolated, non-naturally-occurring peptide as described above can be attached to a heterologous moiety. In certain aspects, the heterologous moiety can be, without limitation, an antimicrobial agent, a therapeutic agent, a prodrug, a peptide, a linker, a scaffold, a protein, an enzyme, a lipid, a biological response modifier, a pharmaceutical agent, a lymphokine, a heterologous antibody or fragment thereof, a detectable label, a polyethylene glycol (PEG), and any combination thereof.

The disclosure further provides an immunogenic composition comprising an isolated, non-naturally-occurring peptide as described above. Moreover, the disclosure provides a method of producing an antibody or antigen-binding fragment thereof that specifically binds to an orthologous filovirus glycoprotein epitope, comprising administering the provided immunogenic composition to an animal, and recovering the antibody or fragment thereof.

Two exemplary binding domains can be derived from the VH and VL antigen binding domains of murine monoclonal antibodies 2D8 and 21D10 (also referred to herein as m2D8 and m21D10), which bind to the RBR across at least five different species of filovirus, e.g., the binding domain can bind to the orthologous epitope as expressed in EBOV, SUDV, MARV, RESTV, and BDBV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 1 and 2 (the VH and VL of 2D8), or SEQ ID NO: 3 and 4 (the VH and VL of 21D10). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 1 and 2, or SEQ ID NO: 3 and 4.

Another exemplary binding domain can be derived from the VH and VL antigen binding domains of murine monoclonal antibody 16G8 (also referred to herein as m16G8), which can bind to the filovirus glycoprotein across three species of filovirus, e.g., the binding domain can bind to the orthologous epitope as expressed in EBOV, SUDV, and BDBV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 5 and 6 (the VH and VL of 16G8). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 5 and 6.

Another exemplary binding domain can be derived from the VH and VL antigen binding domains of murine monoclonal antibody 17C6 (also referred to herein as m17C6), which can bind to the filovirus glycoprotein across four species of filovirus, e.g., the binding domain can bind to the orthologous epitope as expressed in EBOV, SUDV, MARV, and BDBV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 7 and 8 (the VH and VL of 17C6). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 7 and 8.

Another exemplary binding domain can be derived from the VH and VL antigen binding domains of murine monoclonal antibody 8C4 (also referred to herein as m8C4), which can bind to the filovirus glycoprotein across two species of filovirus, e.g., the binding domain can bind to the orthologous epitope as expressed in EBOV and SUDV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 9 and 10 (the VH and VL of 8C4). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 9 and 10.

Another exemplary binding domain can be derived from the VH and VL antigen binding domains of murine monoclonal antibody 4B8 (also referred to herein as m4B8), which can bind to the filovirus glycoprotein across four species of filovirus, e.g., the binding domain can bind to the orthologous epitope as expressed in EBOV, SUDV, RESTV, and BDBV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 11 and 12 (the VH and VL of 4B8). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 11 and 12.

Another exemplary binding domain can be derived from the VH and VL antigen binding domains of murine monoclonal antibody 21B2 (also referred to herein as m21B2), which can bind to the filovirus glycoprotein across at least two species of filovirus, e.g., the binding domain can bind to the orthologous epitope at least as expressed in SUDV and MARV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 13 and 14 (the VH and VL of 21B2). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 13 and 14.

One exemplary binding domain can be derived from the VH and VL antigen binding domains of macaque monoclonal antibody FVM02P, which bind to surface glycoprotein of at least three different species of filovirus, e.g., the binding domain can bind to the orthologous epitope as expressed in EBOV, SUDV, and MARV. I In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 15 and 16 (the VH and VL of FVM02P). I In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 15 and 16.

Another exemplary binding domain can be derived from the VH and VL antigen binding domains of macaque monoclonal antibody FVM03, which can bind to at least the MARV surface glycoprotein. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 17 and 18 (the VH and VL of FVM03). In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 17 and 18.

Another exemplary binding domain can be derived from the VH and VL antigen binding domains of one or more of macaque monoclonal antibodies FVM04, FVM09, FVM11, FVM13, or FVM20, each of which can bind to the filovirus glycoprotein across at least two species of filovirus, e.g., the binding domain can bind to the orthologous epitope as expressed in EBOV and SUDV. In certain aspects the binding domain of this exemplary pan-filovirus binding molecule or fragment thereof can bind to the same orthologous epitope as an antibody or antigen-binding fragment thereof comprising a heavy chain variable region (VH) and light chain variable region (VL) comprising, respectively, the amino acid sequences SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28 (the respective VHs and VLs of FVM04, FVM09, FVM11, FVM13, or FVM20). I In certain aspects the binding domain of this exemplary pan-filovirus binding molecule or fragment thereof can competitively inhibit antigen binding by an antibody or antigen-binding fragment thereof comprising a heavy chain variable region (VH) and light chain variable region (VL) comprising, respectively, the amino acid sequences SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28.

In certain aspects a pan-filovirus binding molecule, e.g., a murine- and/or NHP-derived binding molecule for use in the methods provided herein can be an anti-filovirus antibody or antigen-binding fragment thereof. For example in certain aspects a pan-filovirus binding molecule, e.g., a murine- and/or NHP-derived binding molecule for use in the methods provided herein can comprise a binding domain that comprises VH-CDR1, VH-CDR2, VH-CDR3, VL-CDR1, VL-CDR2, and VL-CDR3 amino acid sequences identical or identical except for four, three, two, or one single amino acid substitutions, deletions, or insertions in one or more CDRs to the CDRs contained in the VH and VL amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

Furthermore, in certain aspects a pan-filovirus binding molecule, e.g., a murine- and/or NHP-derived binding molecule for use in the methods provided herein can comprise a binding domain that comprises VH and VL amino acid sequences at least 85%, 90%, 95%, or 100% identical to reference amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

In certain aspects a pan-filovirus binding molecule, e.g., a murine- and/or NHP-derived binding molecule for use in the methods provided herein can be, e.g., a mouse antibody, a NHP antibody, a humanized antibody, a chimeric antibody, or a fragment thereof. Moreover, the antibody or fragment thereof can be a monoclonal antibody, a component of a polyclonal antibody mixture, a recombinant antibody, a multispecific antibody, or any combination thereof.

In certain aspects a pan-filovirus binding molecule, e.g., a murine- and/or NHP-derived binding molecule for use in the methods provided herein can be a bispecific antibody or fragment thereof that further comprises a second binding domain. Certain bispecific antibodies as provided herein can be engineered to be targeted to the endosomal regions of a filovirus-infected cell. See, e.g., Provisional Patent Appl. Ser. No. 62/019,668, filed, Jul. 1, 2014, which is incorporated herein by reference in its entirety. For example, a Murine- and/or NHP-derived bispecific antibody for use in the methods provided herein can comprise a second binding domain that specifically binds to a filovirus epitope that can be surface exposed and accessible to the second binding domain on a filovirus virion particle. In this aspect, the bispecific antibody can be targeted to the endosomal compartment of an infected cell, where cathepsin enzymes can cleave the mucin-like domain that masks the receptor binding region on native filovirus virion particles, thus opening the receptor-binding region up to a binding domain which can then bind to the virus and neutralize the virus infectivity. In certain aspects, the second binding domain can bind to a surface exposed epitope on a virion particle; for example, the second binding domain can specifically bind to an epitope located in the mucin-like domain, an epitope located in the glycan cap of the filovirus glycoprotein, the tip of the fusion loop of the filovirus glycoprotein, the mucin-like domain, the GP2 fusion domain, or any combination thereof.

In certain aspects a pan-filovirus binding molecule, e.g., a murine- and/or NHP-derived binding molecule for use in the methods provided herein can in certain aspects comprise a heavy chain constant region or fragment thereof. The heavy chain can be a murine constant region or fragment thereof, e.g., a human constant region or fragment thereof, e.g., IgM, IgG, IgA, IgE, IgD, or IgY constant region or fragment thereof. Various human IgG constant region subtypes or fragments thereof can also be included, e.g., a human IgG1, IgG2, IgG3, or IgG4 constant region or fragment thereof.

In certain aspects a pan-filovirus binding molecule, e.g., a murine- and/or NHP-derived binding molecule for use in the methods provided herein can further comprise a light chain constant region or fragment thereof. For example, the light chain constant region or fragment thereof can be a murine constant region or fragment thereof, e.g., a human light chain constant region or fragment thereof, e.g., a human kappa or lambda constant region or fragment thereof.

In certain aspects a pan-filovirus binding molecule, e.g., a murine- and/or NHP-derived binding molecule for use in the methods provided herein can comprise a full-size antibody comprising two heavy chains and two light chains. In other aspects, the binding domain of a pan-filovirus binding molecule, e.g., a murine- and/or NHP-derived binding molecule for use in the methods provided herein can be an Fv fragment, an Fab fragment, an F(ab′)2 fragment, an Fab′ fragment, a dsFv fragment, an scFv fragment, an scFab fragment, an sc(Fv)2 fragment, or any combination thereof.

In certain aspects a pan-filovirus binding molecule, e.g., a murine- and/or NHP-derived binding molecule for use in the methods provided herein can fully or partially neutralize infectivity of the filovirus upon binding of the binding domain to the orthologous epitope on a filovirus.

In certain aspects, a pan-filovirus binding molecule, e.g., a murine- and/or NHP-derived binding molecule for use in the methods provided herein can be conjugated to an antiviral agent, a protein, a lipid, a detectable label, a polymer, or any combination thereof.

Treatment Methods Using Pan-Filovirus Binding Molecules, e.g., Murine- and/or NHP-Derived Binding Molecules as Provided Herein

Methods are provided for the use of pan-filovirus binding molecules, e.g., Murine- and/or NHP-derived binding molecules, e.g., cross-reactive anti-filovirus antibodies or fragments thereof, to treat patients having a disease or condition associated with a filovirus infection, or to prevent, reduce, or manage filovirus-induced virulence in a subject infected with a filovirus.

The following discussion refers to methods of treatment of various diseases and disorders with a pan-filovirus binding molecule, e.g., a murine- and/or NHP-derived binding molecule, e.g., a cross-reactive anti-filovirus antibody or antigen-binding fragment thereof that retains the desired properties of anti-filovirus antibodies provided herein, e.g., capable of specifically binding to and neutralizing filovirus infectivity and/or virulence. In some embodiments, a pan-filovirus binding molecule, e.g., a murine- and/or NHP-derived binding molecule, e.g., a cross-reactive anti-filovirus antibody or antigen-binding fragment thereof for use in the methods provided herein can be a murine, human, or humanized antibody. In some embodiments, the anti-filovirus antibody or antigen-binding fragment thereof comprises a binding domain that binds to the same epitope as, or competitively inhibits binding of, one or more of monoclonal antibodies m2D8, m21D10, m16G8, m17C6, m8C4, m4B8, m21B2, FVM02P, FVM03, FVM04, FVM09, FVM11, FVM13, or FVM20 as provided herein. In some embodiments, the binding domain of an anti-filovirus antibody or antigen-binding fragment thereof as provided herein is derived from one or more of monoclonal antibodies m2D8, m21D10, m16G8, m17C6, m8C4, m4B8, m21B2, FVM02P, FVM03, FVM04, FVM09, FVM11, FVM13, or FVM20 as provided herein. In certain embodiments the binding domain of the derived antibody is an affinity-matured, chimeric, or humanized antibody. In some embodiments a pan-filovirus binding molecule, e.g., a murine- and/or NHP-derived binding molecule, e.g., a cross-reactive anti-filovirus antibody or antigen-binding fragment thereof further comprises a second binding domain that can target the binding domain to the endosome of a virus-infected cell.

In one embodiment, treatment includes the application or administration of a pan-filovirus binding molecule, e.g., a murine- and/or NHP-derived binding molecule, e.g., a cross-reactive anti-filovirus antibody or antigen-binding fragment thereof as provided herein, to a subject or patient, where the subject or patient has been exposed to a filovirus, infected with a filovirus, has a filovirus disease, a symptom of a filovirus disease, or a predisposition toward contracting a filovirus disease. In another embodiment, treatment can also include the application or administration of a pharmaceutical composition comprising a pan-filovirus binding molecule, e.g., a murine- and/or NHP-derived binding molecule, e.g., a cross-reactive anti-filovirus antibody or antigen-binding fragment thereof as provided herein, to a subject or patient, so as to target the pharmaceutical composition to an environment where the Murine- and/or NHP-derived binding molecule can be most effective, e.g., the endosomal region of a virus-infected cell.

In accordance with the methods of the present disclosure, at least one pan-filovirus binding molecule, e.g., a murine- and/or NHP-derived binding molecule as provided herein, e.g., a cross-reactive anti-filovirus antibody or antigen-binding fragment thereof as defined elsewhere herein, can be used to promote a positive therapeutic response. By “positive therapeutic response” is intended any improvement in the disease conditions associated with the activity of the Murine- and/or NHP-derived binding molecule, and/or an improvement in the symptoms associated with the disease. Thus, for example, an improvement in the disease can be characterized as a complete response. By “complete response” is intended an absence of clinically detectable disease with normalization of any previously test results. Such a response can in some cases persist, e.g., for at least one month following treatment according to the methods of the disclosure. Alternatively, an improvement in the disease can be categorized as being a partial response.

Pharmaceutical Compositions and Administration Methods

Methods of preparing and administering a pan-filovirus binding molecule, e.g., a murine- and/or NHP-derived binding molecule, e.g., a cross-reactive anti-filovirus antibody or antigen-binding fragment thereof provided herein, to a subject in need thereof are well known to or are readily determined by those skilled in the art. The route of administration of a pan-filovirus binding molecule, e.g., a murine- and/or NHP-derived binding molecule, e.g., a cross-reactive anti-filovirus antibody or antigen-binding fragment thereof for use in the methods provided herein can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as suitable forms, another example of a form for administration would be a solution for injection, in particular for intravenous or intraarterial injection or drip. In some cases a suitable pharmaceutical composition can comprise a buffer (e.g. acetate, phosphate or citrate buffer), a surfactant (e.g. polysorbate), optionally a stabilizer agent (e.g. human albumin), etc. In other methods compatible with the teachings herein, a pan-filovirus binding molecule, e.g., a murine- and/or NHP-derived binding molecule, e.g., a cross-reactive anti-filovirus antibody or antigen-binding fragment thereof as provided herein can be delivered directly to a site where the binding molecule can be effective in virus neutralization, e.g., the endosomal region of a filovirus-infected cell.

As discussed herein, a pan-filovirus binding molecule, e.g., a murine- and/or NHP-derived binding molecule, e.g., a cross-reactive anti-filovirus antibody or antigen-binding fragment thereof provided herein, can be administered in a pharmaceutically effective amount for the in vivo treatment of diseases or disorders associated with filovirus infection. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a pan-filovirus binding molecule, e.g., a murine- and/or NHP-derived binding molecule, e.g., a cross-reactive anti-filovirus antibody or antigen-binding fragment thereof means an amount sufficient to achieve effective binding to a target and to achieve a benefit, e.g., to ameliorate symptoms of a disease or condition or to detect a substance or a cell. Suitable formulations for use in the therapeutic methods disclosed herein can be described in Remington's Pharmaceutical Sciences (Mack Publishing Co.) 16th ed. (1980).

The amount of a pan-filovirus binding molecule, e.g., a murine- and/or NHP-derived binding molecule, e.g., a cross-reactive anti-filovirus antibody or antigen-binding fragment thereof that can be combined with carrier materials to produce a single dosage form will vary depending upon the subject treated and the particular mode of administration. The composition can be administered as a single dose, multiple doses or over an established period of time in an infusion. Dosage regimens also can be adjusted to provide an optimum response (e.g., a therapeutic or prophylactic response).

In keeping with the scope of the present disclosure, a pan-filovirus binding molecule, e.g., a murine- and/or NHP-derived binding molecule, e.g., a cross-reactive anti-filovirus antibody or antigen-binding fragment thereof for use in the methods provided herein can be administered to a human or other animal in accordance with the aforementioned methods of treatment in an amount sufficient to produce a therapeutic effect. A pan-filovirus binding molecule, e.g., a murine- and/or NHP-derived binding molecule, e.g., a cross-reactive anti-filovirus antibody or antigen-binding fragment thereof provided herein can be administered to such human or other animal in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of a pan-filovirus binding molecule, e.g., a murine- and/or NHP-derived binding molecule, e.g., a cross-reactive anti-filovirus antibody or antigen-binding fragment thereof, that when administered brings about a positive therapeutic response with respect to treatment of a patient with a disease or condition to be treated.

Therapeutically effective doses of the compositions disclosed herein, for treatment of diseases or disorders associated with filovirus infection, vary depending upon many different factors, including means of administration, target site, physiological state of the patient, whether the patient is human or an animal, other medications administered, and whether treatment is prophylactic or therapeutic. Usually, the patient is a human, but non-human mammals including non-human primates can also be treated. Treatment dosages can be titrated using routine methods known to those of skill in the art to optimize safety and efficacy.

The amount of a pan-filovirus binding molecule, e.g., a murine- and/or NHP-derived binding molecule, e.g., a cross-reactive anti-filovirus antibody or antigen-binding fragment thereof to be administered can be readily determined by one of ordinary skill in the art without undue experimentation given this disclosure. Factors influencing the mode of administration and the respective amount of a pan-filovirus binding molecule, e.g., a murine- and/or NHP-derived binding molecule, e.g., a cross-reactive anti-filovirus antibody or antigen-binding fragment thereof include, but are not limited to, the severity of the disease, the history of the disease, and the age, height, weight, health, and physical condition of the individual undergoing therapy. Similarly, the amount of a pan-filovirus binding molecule, e.g., a murine- and/or NHP-derived binding molecule, e.g., a cross-reactive anti-filovirus antibody or antigen-binding fragment thereof to be administered will be dependent upon the mode of administration and whether the subject will undergo a single dose or multiple doses of this agent.

This disclosure also provides for the use of a pan-filovirus binding molecule, e.g., a murine- and/or NHP-derived binding molecule, e.g., a cross-reactive anti-filovirus antibody or antigen-binding fragment thereof in the manufacture of a medicament for treating, preventing, or managing a disease or disorder associated with filovirus infection, e.g., hemorrhagic fever.

### EXAMPLES

We have employed an engineered, MLD-deleted form of filovirus glycoproteins was used as immunogen to elicit broadly reactive filoviral monoclonal antibodies in rhesus macaques and mice.

### Example 1: Characterization of Pan-Filovirus Antibodies

ELISA to Test Purified Mouse Monoclonal Antibodies:

Purified GPddmuc or GPdTM or cathepsin-cleaved GP were immobilized on 96-well Nunc MaxiSorp plates (ThermoFisher Scientific) and incubated with serial dilutions of purified mouse monoclonal antibodies. Bound antibodies were detected using an HRP-conjugated anti-mouse secondary antibody (KPL) and TMB substrate (Life Technologies). Absorbance values determined at 650 nm were transformed using Softmax® 4 parameter curve-fit (Molecular Devices). Half maximal effective concentration (EC50) value at the inflection point of the curve is reported. The results are provided in Table 2.

TABLE 2
Relative binding of anti filovirus monoclonal antibodies to GPddmuc and GPdTM as
well as cathepsin-cleaved EBOV GP as determined by ELISA. The values represent the
concentration of the antibody that results in 50% maximum binding (EC₅₀).

| | | | | | | | | | ELISA |
|---|---|---|---|---|---|---|---|---|---|
| | | | | | | | | | EC50 |
| | | | | | | | | | μg/ml |
| | | | | | | ELISA EC50 μg/ml | | | (Cleaved |
| | ELISA EC50 μg/ml (GPdTM) | | | | | (GPddmuc) | | | EBOV |
| Mab | EBOV | SUDV | MARV | RESTV | BDBV | EBOV | SUDV | MARV | GP) |
| 2D8 | >10 | >10 | 0.0445 | NT | NT | 26.2 | >10 | 0.0573 | 0.72 |
| 21D10 | 1.22 | >10 | 4.22 | 1.84 | 1.07 | 2.22 | 1.33 | 1.8 | 0.12 |
| 16G8 | >10 | >10 | ND | >10 | 0.5 | 1.23 | 0.22 | ND | NT |
| 17C6 | 0.04 | 2 | ND | 1.56 | >10 | 0.03 | 0.03 | ND | NT |
| 8C4 | 0.26 | 0.15 | ND | ND | >10 | 0.21 | 0.06 | ND | NT |
| 4B8 | 0.088 | 0.088 | ND | 0.157 | 0.033 | 0.69 | 0.095 | ND | NT |
| 21B2 | ND | >10 | 0.0688 | NT | NT | >10 | >10 | 0.0307 | NT |

ND = Not Detected
NT = Not Tested

Determination of Relative Binding of Macaque-Human Chimeric Antibodies to Filovirus Glycoproteins or Virus-Like Particles (VLP).

Relative binding of the antibodies to different filovirus glycoproteins and VLPs was determined by ELISA at various concentrations of the antibodies and the effective concentration at 50% maximal binding (EC₅₀) determined. Purified GPddmuc, GPdTM, or VLPs from the three filovirus species (SUDV, EBOV, and MARV) were immobilized on 96-well Nunc MaxiSorp plates (ThermoFisher Scientific) and incubated with serial dilutions of the macaque-human chimeric antibodies. Bound antibodies were detected using an HRP-conjugated anti-mouse secondary antibody (KPL) and TMB substrate (Life Technologies). Absorbance values determined at 650 nm were transformed using Softmax® 4 parameter curve-fit (Molecular Devices). Table 3 shows the EC₅₀ values.

In Vitro Neutralization of Live Filovirus by EBOV and SUDV in Microneutralization Assays:

Vero E6 cells seeded at a density of 4.0E+05 cells/well were incubated for 24 hours at 37° C. with 5% CO₂. Virus stock (1.20E+04 pfu) was incubated with antibody for 1 hour then split equally into 3 wells (replicates) containing cells to achieve 4.0E+03 pfu per well. The inoculum was removed after one hour at 37° C. with 5% CO₂ and replaced with fresh media. For EBOV microneutralization, cells were permeabilized with methanol then fixed using 10% phosphate buffered formalin for cell-based ELISA. Rabbit polyclonal antibody was used for the detection of EBOV matrix protein (VP40). Monoclonal antibody known to neutralize EBOV was used as a positive control. For SUDV microneutralization, cells were fixed using 10% phosphate buffered formalin for cell-based ELISA. Rabbit polyclonal antibody was used for the detection of SUDV glycoprotein (GP). Monoclonal antibody known to neutralize SUDV was used as a positive control. The assays utilized a luminescent substrate (SuperSignal ELISA Pico, Pierce) for detection in the cell-based ELISA assays. As shown in Table 4, several macaque antibodies showed significant neutralization of both EBOV and SUDV.

### Example 2: Epitope Mapping

Epitope Mapping of Pan-Ebola Monoclonal Antibodies FVM09 and FVM11:

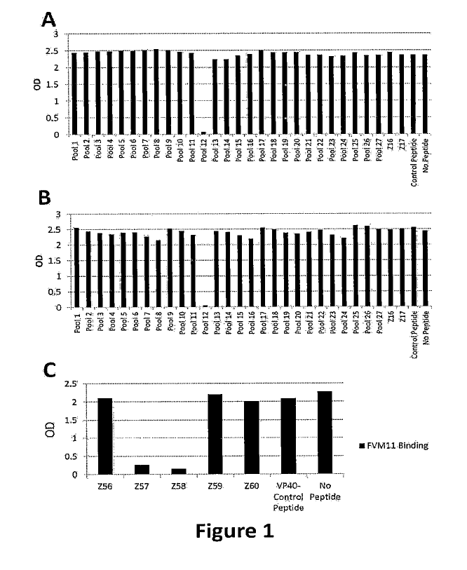

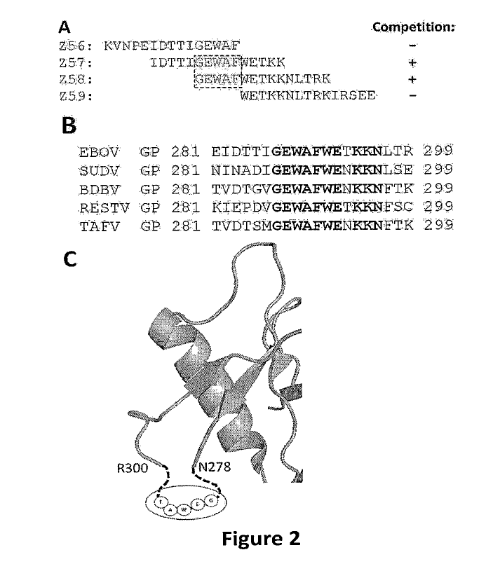

Western blot analysis demonstrated that FVM09 and FVM11 can detect filovirus glycoproteins from four different ebolavirus species, EBOV, SUDV, BDBV, and RESTV in denatured form indicating that the antibody recognizes a linear epitope (data not shown). To identify the linear epitope a competition assay was performed using overlapping peptides (15-mers with 10 amino acid overlap) spanning the whole EBOV glycoprotein sequence. Initially 27 pools of five consecutive peptides were prepared. Each pool was pre-incubated with FVM09 or FVM11 at a high molar excess of peptide to antibody. A control, irrelevant peptide as well as no peptide controls were included. The pre-incubated antibodies were added to plates coated with 100 ng/well of purified EBOV GP and incubated for 1 hour. After washing the unbound, the bound antibodies were detected using an HRP-conjugated anti-human polyclonal antibody. Data for this experiment are shown in FIG. 1. Based on these data the epitope was mapped to a region spanning the amino acids 286-300 of EBOV with the sequence motif of GEWAF as the core of the epitope (FIG. 2). This region is highly conserved among Ebola species glycoproteins (FIG. 2B).

The FVM09/FVM11 epitope is located in a domain known as the glycan cap (Lee, et al., 2008, Nature, 454 (7201):177-182) within a disordered loop (amino acids 279-298) connecting β-17 and β-18 (Lee, et al., 2008, Nature, 454 (7201):177-182).

Epitope Mapping of FVM02P:

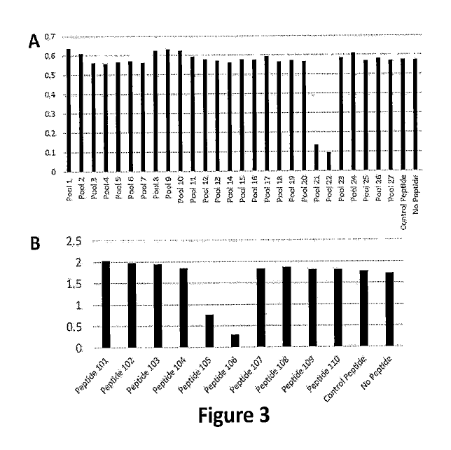

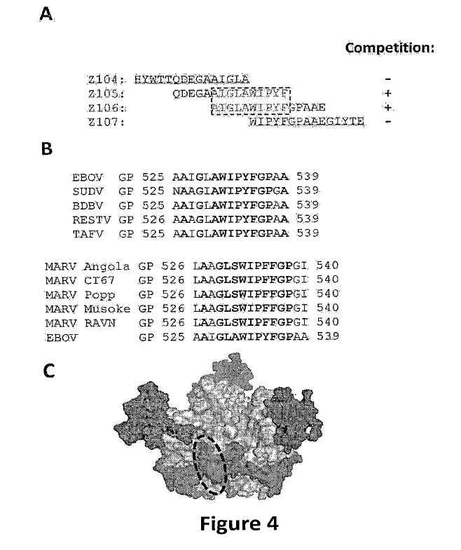

Similar to FVM09 and FVM11, the epitope for another generally linear macaque antibody, FVM02P was identified using a competition assay (FIG. 3). The epitope was mapped to a region spanning the amino acids 525-540 of EBOV with the sequence motif of AIGLAWIPYF as the core of the epitope (FIG. 4). This region is highly conserved among Ebola species as well as the glycoproteins of strains of marburgvirus (FIG. 4B). The epitope is located at the tip of the fusion loop of the filovirus glycoprotein (FIG. 4C), a functionally critical site for viral fusion with host cell membranes (Lee, et al., 2008, Nature, 454 (7201):177-182).

In contrast to FVM02P, FVM09, and FVM11, two other macaque derived antibodies FVM04 and FVM20 appear to bind to generally conformational epitopes, as the binding was lost upon denaturation of antigens.

### Example 3: Efficacy of Mouse Monoclonal and Macaque-Derived (Macaque-Human Chimeric) Anti-Filovirus Monoclonal Antibodies

Method:

Efficacy of anti-filovirus antibodies was tested in mouse models of Ebola and Marburg infections. Groups of 5 or 10 BALB/c mice were infected with 1000 PFU of mouse adapted EBOV (MA-EBOV) or mouse adapted MARV (MA-MARV) by intraperitoneal (i.p.) injection. Different dosing regimens where used in various experimental groups. Most groups received two doses of antibodies by i.p. route with the first dose administered within 1-2 hour post infection and the second dose delivered on day 3 post infection. In some experimental groups antibody was only given on day 3 post infection.

Results:

Efficacy of FVM04 and FVM20:

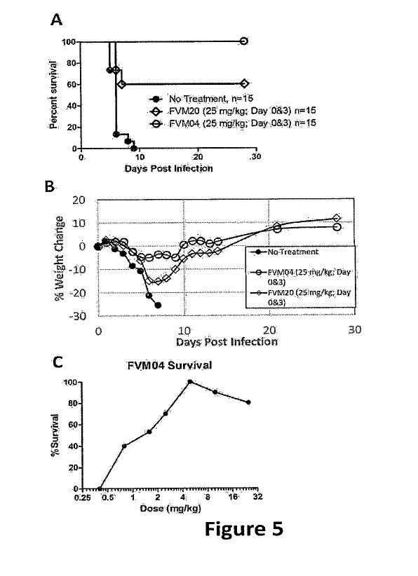

Treatment of mice (groups of 5 mice) with 25 mg/kg of FVM04 on days 0 and 3 resulted in 100% protection against lethal challenge with MA-EBOV, while treatment with FVM20 led to 40% protection (FIG. 5A). In a repeat experiment, groups of 10 mice were challenged and given the antibody either on day 0 and 3 or only on day 3. As shown in Table 5, 10 out of 10 mice treated with FVM04 and 7 out of 10 mice treated with FVM20 survived the challenge when the antibodies were given on day 0 and day 3. In contrast all control mice died upon challenge. When FVM04 was given on day 3 only, 4 out of 10 mice still survived the challenge, while treatment on day 4 did not result in survival.

Efficacy of m4B8:

As shown in Table 5, treatment of mice (groups of 5 mice) with 25 mg/kg of m4B8 on days 0 and 3 resulted in 100% protection against lethal challenge with MA-EBOV. In a repeat study (Study 2), mice received m4B8 at 30 mg/kg only once on day 3 post challenge and 80% of the mice survived the challenge (Table 5).

Efficacy of Glycan Cap Binding Antibodies m8C4 and FVM09:

As shown in Table 5, in two studies combined, m8C4 protected 7 out of 15 mice when mice were treated with 25 mg/kg on days 0 and 3. FVM09 protected 10 out of 15 mice in the two studies combined.

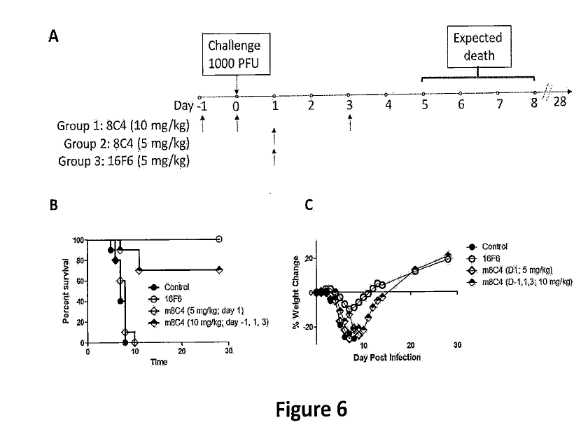

Efficacy of m8C4 was also tested in a novel model of Sudan virus infection in AG129 mice (Dye, J., et al., J. Inf. Dis., in press). As shown in FIG. 6, treatment of mice with 10 mg/kg of m8C4 on days −1, +1, and +3 relative to the challenge with 1000 PFU of wild type SUDV resulted in significant protection. This is the first time a single antibody is shown to protect against two different Ebola species.

Efficacy of FVM02P: As shown in Table 5, in two studies combined FVM02P protected 8 out of 15 mice challenged with Ebola virus. The efficacy of FVM02P (which is a pan filovirus antibody) was also examined in Marburg virus challenge model using mouse-adapted MARV. In this study FVM02 treatment on days 0 and 3 (25 mg/kg) resulted in protection of 4 out of 15 mice challenged with MARV.

### Example 4: In Vivo Studies with Combinations of Monoclonal Antibodies

Combinations of antibodies targeting different epitopes were tested for efficacy in mouse model of EBOV infection, as follows.

Two Antibodies Binding to EBOV Glycoprotein Glycan Cap Region:

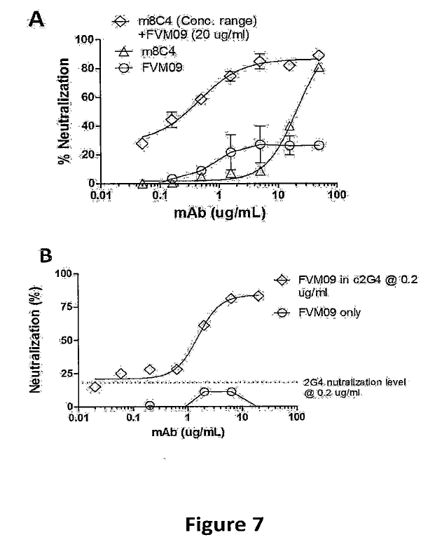

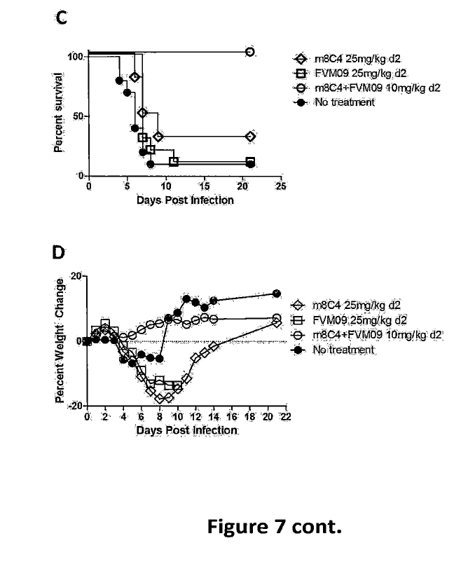

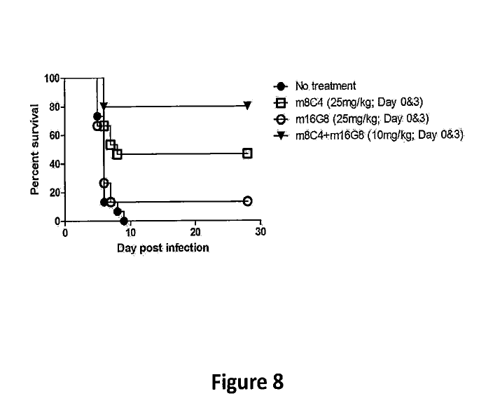

m8C4 (conformational glycan cap binder) was combined with FVM09 (recognizing a linear epitope in glycan cap). The result of the study showing enhanced protection is shown in FIG. 7A. Mice receiving a combination of m8C4 and FVM09 at 10 mg/kg each showed no weight loss, while mice treated with either antibody alone at a higher dose of 25 mg/kg lost significant weight after challenge (FIG. 7B). The data were further confirmed in a second study (Table 5).

Combination of a Glycan Cap Binder with an Antibody Binding to a Conformational Epitope.

The glycan cap binder m8C4 was combined with the pan-Ebola m16G8, an antibody that binds to a conformational epitope. As shown in FIG. 8, in the first study, the two antibodies administered singly did not provide significant protection against lethal challenge when used at 25 mg/kg. However, combination of the two antibodies at a lower dose of 10 mg/kg each resulted in 80% protection from lethal EBOV challenge. These results were further confirmed in Study 2 (Table 5). Moreover, in Study 2, the combination of m8C4 and m16G8 at 15 mg/kg each also protected 30% of mice when given only on day 3, a time point very close to death from EBOV challenge.

Combination of a Glycan Cap Binder with an Antibody Targeting an Epitope within the Fusion Loop.

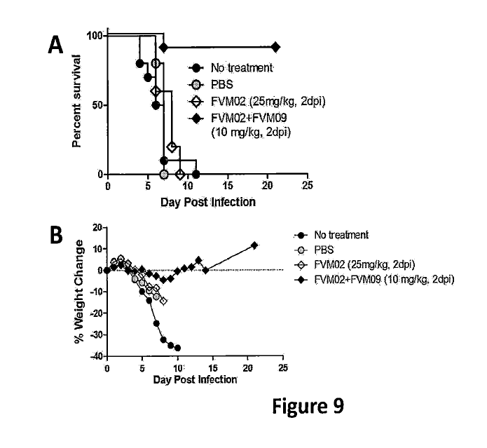

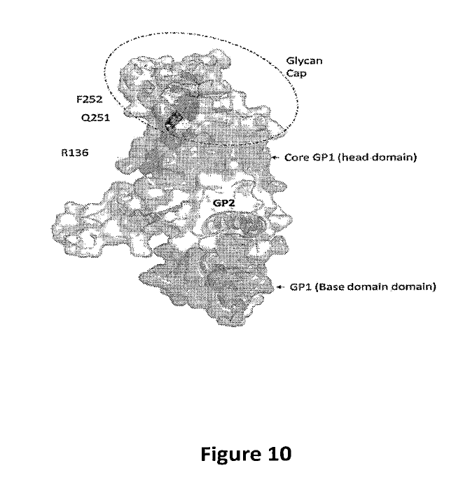

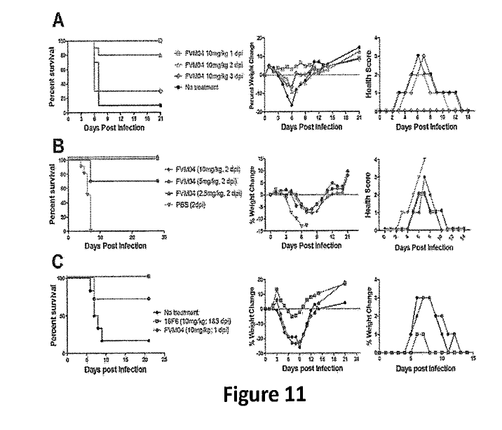

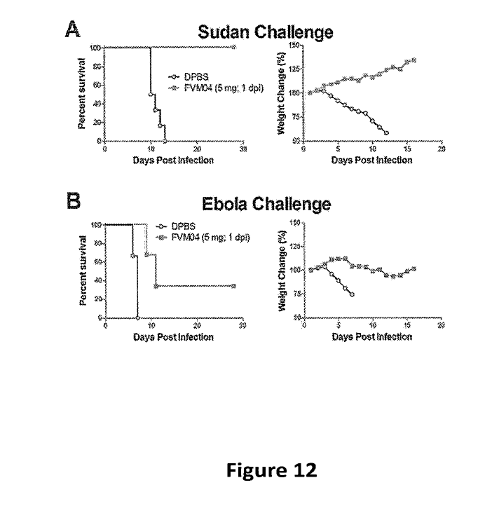

A combination treatment with the glycan cap binder FVM09 with the fusion loop binder FVM02P was investigated. In Study 1 (FIG. 9A), the FVM09 provided 60% protection and FVM02 protected at 20% when given at 25 mg/kg on days 0 and 3, but the combination given at a lower dose of 10 mg/kg each resulted in 80% protection. Moreover, the mice receiving the combination at a lower dose showed no weight loss (FIG. 9B). The findings were further confirmed in Study 2 (Table 5).

TABLE 5
Survival of mice upon treatment with monoclonal Pan-Ebola antibodies after challenge
with MA-EBOV.

| | | | | Treatment | |
|---|---|---|---|---|---|
| | | | | Time | |
| Study# | N | Antibody | Dose | Point | % Survival |
| Study 1 | 5 | m2D8 | 25 mg/kg | Dy 0&8 | 0% |
| Study 1 | 5 | m21D10 | 25 mg/kg | Day 0&3 | 0% |
| Study 2 | 5 | h21D10v1.4 | 30 mg/kg | Day 0&3 | 20% |
| Study 1 | 5 | m21B2 | 25 mg/kg | Day 0&3 | 40% |
| Study 1 | 5 | m2D8 + m21D10 + m21B2 | 10 mg/kg each | Day 0&3 | 0% |
| Study 1 | 5 | m8C4 | 25 mg/kg | Day 0&3 | 0% |
| Study 2 | 10 | m8C4 | 25 mg/kg | Day 0&3 | 70% |
| Study 1 | 5 | m17C6 | 25 mg/kg | Day 0&3 | 20% |
| Study 1 | 5 | m16G8 | 25 mg/kg | Day 0&3 | 0% |
| Study 2 | 10 | m16G8 | 25 mg/kg | Day 0&3 | 20% |
| Study 1 | 5 | m8C4 + m16G8 | 10 mg/kg each | Day 0&3 | 80% |
| Study 2 | 10 | m8C4 + m16G8 | 15 mg/kg each | Day 0&3 | 80% |
| Study 2 | 10 | m8C4 + m16G8 | 15 mg/kg each | Day 3 | 30% |
| Study 1 | 5 | m4B8 | 25 mg/kg | Day 0&3 | 100% |
| Study 2 | 5 | m4B8 | 30 mg/kg | Day 3 | 80% |
| Study 1 | 5 | FVM02P | 25 mg/kg | Day 0&3 | 20% |
| Study 2 | 10 | FVM02P | 25 mg/kg | Day 0&3 | 60% |
| Study 1 | 5 | FVM03 | 25 mg/kg | Day 0&3 | 20% |
| Study 1 | 5 | FVM04 | 25 mg/kg | Day 0&3 | 100% |
| Study 2 | 10 | FVM04 | 25 mg/kg | Day 0&3 | 100% |
| Study 2 | 10 | FVM04 | 30 mg/kg | Day 3 | 40% |
| Study 1 | 5 | FVM09 | 25 mg/kg | Day 0&3 | 60% |
| Study 2 | 10 | FVM09 | 25 mg/kg | Day 0&3 | 70% |
| Study 2 | 10 | FVM09 | 30 mg/kg | Day 3 | 0% |
| Study 2 | 10 | FVM02 + FVM09 | 15 mg/kg each | Day 0&3 | 100% |
| Study 1 | 5 | m8C4 + FVM09 | 10 mg/kg each | Day 0&3 | 100% |
| Study 2 | 10 | m8C4 + FVM09 | 10 mg/kg each | Day 0&3 | 100% |
| Study 2 | 10 | m8C4 + FVM09 | 15 mg/kg each | Day 3 | 0% |
| Study 2 | 10 | FVM09 | 30 mg/kg | Day 4 | 0% |
| Study 2 | 10 | FVM04 | 30 mg/kg | Day 4 | 0% |
| Study 2 | 10 | m8C4 + FVM09 | 15 mg/kg each | Day 4 | 0% |
| Study 1 | 5 | FVM13 | 25 mg/kg | Day 0&3 | 20% |
| Study 1 | 5 | FVM02P + FVM09 | 10 mg/kg each | Day 0&3 | 80% |
| Study 1 | 5 | FVM20 | 25 mg/kg | Day 0&3 | 40% |
| Study 2 | 10 | FVM20 | 25 mg/kg | Day 0&3 | 70% |
| Study 1 | 10 | No Treatment | N/A | N/A | 0% |
| Study 2 | 10 | No treatment | N/A | N/A | 0% |
| Study 1 | 5 | Positive Control (6D8) | 25 mg/kg | Day 0&3 | 100% |
| Study 2 | 10 | Positive Control (6D8) | 25 mg/kg | Day 0&3 | 100% |

TABLE 6
Antibody Sequences

| | SEQ | | SEQ | |
|---|---|---|---|---|
| Antibody | ID | Heavy Chain | ID | Light Chain |
| m2D8 | 1 | EVQLQESGGELVRPGTSVKIS | 2 | DVVMTQTPLTLSVTIGQPASI |
| | | CKASGYTFTNFWLGWIKERPG | | SCKSSQSLLYSDGETYLNWLL |
| | | HGLEWIGDIYPGGHYTNYNEN | | QRPGQSPKRLIYLVSKLDSGV |
| | | FKGKATLTADTSSNTAYMQVS | | PDRFTGSGSGTDFTLKISRVE |
| | | SLTSEDSAVYFCFTQYYDMDY | | AEDLGVYYCWQGTHFPQTFGG |
| | | WGQGTSVTVSS | | GTKLEIKR |
| m22D10 | 3 | EVQLQESGPELVKPGASVKMS | 4 | DIVMTQAAFSNPVTLGTSASI |
| | | CKASGYTFTDYYMNWVKQSHG | | SCRSSKSLLHSNGITYLYWYL |
| | | KSLEWIGDINPYNGDTTYNQK | | QKPGQSPXLLIYQMSNLASGV |
| | | FKGKATLTVDKSSSTAYMQLN | | PDRFSSSGSGTDFTLRISRVE |
| | | SLTSEDSAVYYCASGYEGDYW | | AEDVGVYYCAQNLELPTFGSG |
| | | GQGTTLTVSS | | TKLEIKR |
| m16G8 | 5 | EVQLQESGPGLVKPGASVKIS | 6 | DIQMTQSPASLSASVGETVTI |
| | | CKASGYSFTGYYMNWVKQSPE | | TCRASENIYSNLAWYQQKQGK |
| | | KSLEWIGEINPSTGGTTYNQK | | SPQLLVNAATNLADGVPSRFS |
| | | FKAKATLTVDKSSSTAYMQLK | | GSGSGTQFSLKINSLQPEDFG |
| | | SLTSEDSAVYYCVRYSHYYGS | | SYYCQHFYVTPYMFGSGTKLE |
| | | GSWYFDYWGTGTTVTVSS | | IKR |
| m17C6 | 7 | LVQLQLSGPGLVQPSQSLSIT | 8 | DIQMTQTTSSLSASLGDRVTI |
| | | CTVSGFSLTSYGVHWVRQSPG | | SCRASQDISNYLNWYQQKPDG |
| | | KGLEWLGVIWSGGSTDYNAAF | | TVKLLIYYTSRLHSGVPSRFS |
| | | ISRLSISKDNSKSQVFFKMNS | | GSGSGTDFSLTISNQEPEDIA |
| | | LQADDTAIYYCARNWFDYDNY | | TYYCQQYSNLPWTFGGGTKLE |
| | | YSMDYWGQGTSVTVSS | | IKR |
| m8C4 | 9 | EVQLQESGPELKKPGETVKIS | 10 | DIQMTQSPSSLSASLGGKVTI |
| | | CKASGYTFTTYGMSWVKQAPG | | TCKASQDINKNIAWYQIIKPG |
| | | KGLKWMGRINTYSGVPTYADE | | KGPRLLIWYKSALQPGIPSRF |
| | | FKGRFAFSLENSASTAYLQIN | | SGSGSGRDYSFSISNLEPEDI |
| | | NLKNEDTATYFCARGDGGHWF | | ATYSCLQYDNLPPTFGGGTKL |
| | | ANWGQGTLVTVSA | | EIKR |
| m4B8 | 11 | EVQLQESGPGILQPSQTLSLT | 12 | KFPSQLLLLLLFGIPGDRVTI |
| | | CSFSGFSLRTLGMGVGWTRQS | | TCKASEDIYNRLAWYQQKPGN |
| | | SGKGLEWLAHIWWSDENYYNP | | APRLLISGATSLETGVPSRFS |
| | | ALKSRLTISKDTSKNQVFLRI | | GSGSGKDYTLSITSLQTEDVA |
| | | ANVDTAETATYYCARIAYDGY | | TYYCQQYWTTPWTFGGGTKLE |
| | | FDYWGQGTSLTVSS | | IKR |
| m21B2 | 13 | EVQLQESGAELVRPGASVTLS | 14 | DIVMSQSPSSLAVSAGEKVTM |
| | | CKTSGYTFTDYEMHWVKQTPV | | SCKSSQSLFNSRSRKNYLAWY |
| | | HGLEWIGAIDPETGGTAYNQK | | QLKPGQSPKLLIYWASTRESG |
| | | FKGKATLTADKSSSTAYMELS | | VPDRFTGSGSGTDFTLTISSV |
| | | RLTSEDSAVYYCTGNYATDYW | | QAEDLAVYYCKQSNYLWTFGG |
| | | GQGTSVTVSS | | GTKLEIKR |
| FVM02P: | 15 | EVQLVESGGGLVQPGGSLRLS | 16 | DIVLTQSPLSLPVTPGEPASI |
| | | CAASGFTGFTFSDYAFYWVRQ | | SCRSSQSLLHSGGKTYLYWYL |
| | | APGKGLEWVGFIRGKAYGGTA | | QKPGQSPQLLIHEVSNRASGV |
| | | DYAASVKGRFTISRDNSKNTA | | PDRFSGSGSGTDFTLKISRVE |
| | | YLQMSSLKTEDSAVYYCTSQG | | AEDVGVYYCMQGIQLPLTFGG |
| | | VTVATPYHWGQGVLVTVSS | | GTKVEIKRTV |
| FVM03: | 17 | QVQLQESGPVLVKPSETLSLT | 18 | EIVLTQSPDSLGVSLGERVTI |
| | | CAVSGYSISSAYAWSWIRQPP | | MCKSSQSLLYSSNNKNYLAWY |
| | | GKGLEWVGYVGSSSDYNPSLK | | QQKPGQAPKLLIYWASTRESG |
| | | SRVTISRDTSKNRIFLNLRSL | | VPNRFSGSGSGTDFTLTISGL |
| | | TAADTAVYYCARDRANNSMDV | | QAEDVAVYYCQQYYSTPLTFG |
| | | WGRGVLVTVSS | | QGTKVEIKRTV |
| FVM04: | 19 | EVQLVQSGGGLVQPGGSMRLS | 20 | DVVMTQSPSFLSASVGDRVTI |
| | | CEASGLSLSDYFMHWVRQAQG | | TCRASQDITINLNWFQHKPGK |
| | | KGLEWIGLIQTKAFTYKTEYP | | APKRLIYVASRLERGVPSRFS |
| | | AAVKGRFTISRDDSKNTLYLQ | | GSGSGTEFTLTISSLQPEDFA |
| | | MSSLKPEDTALYYCIAVTPDF | | TYYCQQYNNYPLTFGPGTKLD |
| | | YYWGQGVLVTVSS | | IKRTV |
| FVM09: | 21 | EVQLVQSGAEVKRPGESLTIS | 22 | QLVLTQPASLSASPGASASLT |
| | | CKTSGYSFTDSWVAWVRQMPG | | CTFSGGINVAGYNILWYQQKA |
| | | KGLEWLGSIYPGDSETKYNPS | | GSPPRYLLRYKSDSTKDQGSG |
| | | FQGHVTISADKSVTTTYLKWS | | VPSRFSGSKDASANTGILRIS |
| | | SLKASDTATYYCVKGSETWGQ | | GLQSEDEADYYCAIGHSSGWI |
| | | GVLVTVSS | | FGGGTRLTVL |
| FVM13: | 23 | EVQLVQSGAEVKRPGESLKIS | 24 | KPMLTQPTSLSASPGASASLT |
| | | CKTSGYTFTSSWISWVRQMPG | | CTFSGGINVAGYNILWYQQKA |
| | | KGLEWLGSIYPGDSDTRYNPS | | GSPPRYLLRYKSDSTKDQGSG |
| | | FQGHVTISADTSIITTHLQWS | | VPSRFSGSKDASANTGILRIS |
| | | SLKASDTATYYCVKGSETWGQ | | GLQSEDEADYYCAIGHSSGWI |
| | | GVLVTVSS | | FGGGTRLTVL |
| FVM20: | 25 | EVQLVESGAEVKRPGESLTIS | 26 | NFMLTQSASLSASPGASASLT |
| | | CKTSGYSFTGSWISWVRQMPG | | CTFSGGINVAGYHIFWYQQNP |
| | | KGLEWLGSIYPGDSETKYNPS | | GSPPRYLLRYKSDSEKDQGAG |
| | | FQGHVTISADKSVTTTYLKWS | | VPSRFSGSKDASANTGILRIS |
| | | SLKASDTATYYCVKGSETWGQ | | GLQSEDEADYYCAIGHSSGWI |
| | | GVLVTVSS | | FGGGTRLTVL |

The breadth and scope of the present disclosure should not be limited by any of the above-described exemplary embodiments, but should be defined only in accordance with the following claims and their equivalents.

1. A method for preventing, treating, or managing a filovirus infection in a subject, comprising administering to a subject in need thereof an effective amount of at least one binding molecule comprising a binding domain that specifically binds to an orthologous filovirus glycoprotein epitope, wherein the binding domain specifically binds to the epitope on two or more filovirus species or strains.

2. The method of claim 1, wherein the binding domain binds to the orthologous epitope as expressed in two or more, three or more, four or more, or five or more of Marburg virus (MARV), Ravn virus (RAVV), Tai Forest virus (TAFV), Reston virus (RESTV), Sudan virus (SUDV) Ebola virus (EBOV), Bundibugyo virus (BDBV), or any strain thereof.

3. The method of claim 1, wherein the ability of the binding molecule to prevent, treat, or manage a filovirus infection can be measured in a model comprising administering the binding molecule to a group of mice and challenging mice with a mouse-adapted (MA) filovirus before, at the same time as, or after administering the binding molecule to the mice.

4. The method claim 3, wherein the MA-filovirus is a MA ebolavirus (MA-EBOV), a MA-marburgvirus (MA-MARV), or a combination thereof.

5. The method of claim 3, wherein the mice are monitored for increased survival time, decreased weight loss, or a combination thereof as compared to control mice.

6. The method of claim 1, wherein the at least one binding molecule binds to the same orthologous epitope as the murine monoclonal antibody m2D8, m21D10, m16G8, m17C6, m8C4, m4B8, or m21B2, the macaque monoclonal antibody FVM02P, FVM03, FVM04, FVM09, FVM11, FVM13, or FVM20, or any combination thereof.

7. The method of claim 1, wherein the at least one binding molecule can bind to the same orthologous epitope as an antibody or antigen-binding fragment thereof comprising a heavy chain variable region (VH) and light chain variable region (VL) comprising the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively, or any combination thereof.

8. The method of claim 1, wherein the binding molecule is an antibody or antigen-binding fragment thereof.

9. The method of claim 8, wherein the subject is administered an effective amount of an antibody or antigen-binding fragment thereof comprising VL-CDR1, VL-CDR2, VL-CDR3, VH-CDR1, VH-CDR2, and VH-CDR3 amino acid sequences identical or identical except for four, three, two, or one single amino acid substitutions, deletions, or insertions to the CDRs contained in the VH and VL sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

10. The method of claim 8, wherein the subject is administered an effective amount of an antibody or antigen-binding fragment thereof comprising VH and VL amino acid sequences at least 85%, 90%, 95%, or 100% identical to reference amino acid sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

11. The method of claim 1, wherein the subject is administered an effective amount of a combination of at least two, at least three, at least four, or more binding molecules, wherein each binding molecule comprises a binding domain that specifically binds to an orthologous filovirus glycoprotein epitope, wherein the binding domain specifically binds to the epitope on two or more filovirus species or strains.

12. The method of claim 11, wherein the combination of binding molecules can prevent, treat, or manage filovirus infection in the subject with a potency that is greater than the additive potency of the binding molecules when administered individually.

13. The method of claim 12, wherein the potency levels of the ability of the combined binding molecules to prevent, treat, or manage a filovirus infection can be measured in a model comprising administering the binding molecules to a group of mice and challenging mice with a MA-filovirus before, at the same time as, or after administering the binding molecule to the mice.

14. The method claim 13, wherein the MA-filovirus is MA-EBOV, MA-marburgvirus MA-MARV, or a combination thereof.

15. The method of claim 12, wherein potency is measured as increased survival time, decreased weight loss, or a combination thereof in the mouse model.

16. The method of claim 11, wherein the at least two, at least three, at least four, or more binding molecules bind to the same orthologous epitope or epitopes as at least two, at least three, at least four, or more of the murine monoclonal antibodies m2D8, m21D10, m16G8, m17C6, m8C4, m4B8, or m21B2, the macaque monoclonal antibodies FVM02P, FVM03, FVM04, FVM09, FVM11, FVM13, or FVM20, or any combination thereof.

17. The method of claim 11, wherein the at least two, at least three, at least four, or more binding molecules can bind to the same orthologous epitope or epitopes as an antibody or antigen-binding fragment thereof comprising a heavy chain variable region (VH) and light chain variable region (VL) comprising the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

18. The method of claim 11, wherein the at least two, at least three, at least four, or more binding molecules are antibodies or antigen-binding fragments thereof.

19. The method of claim 18, wherein the subject is administered an effective amount of a combination of at least two, at least three, at least four, or more antibodies or antigen binding fragments thereof comprising VL-CDR1, VL-CDR2, VL-CDR3, VH-CDR1, VH-CDR2, and VH-CDR3 amino acid sequences identical or identical except for four, three, two, or one single amino acid substitutions, deletions, or insertions to the CDRs contained in the VH and VL sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

20. The method of claim 18, wherein the subject is administered an effective amount of a combination of at least two, at least three, at least four, or more antibodies or antigen binding fragments thereof comprising VH and VL amino acid sequences at least 85%, 90%, 95%, or 100% identical to reference amino acid sequences SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, or SEQ ID NO: 27 and SEQ ID NO: 28, respectively.

21. The method of claim 18, wherein the combination at least two, at least three, at least four, or more antibodies or antigen-binding fragments thereof comprises monoclonal antibodies m8C4 and FVM09.

22. The method of claim 18, wherein the combination at least two, at least three, at least four, or more antibodies or antigen-binding fragments thereof comprises monoclonal antibodies m8C4 and m16G8.

23. The method claim 18, wherein the combination at least two, at least three, at least four, or more antibodies or antigen-binding fragments thereof comprises monoclonal antibodies FVM09 and FVM02P.

24. The method of claim 1, wherein the orthologous epitope is or epitopes are in the GP-1 subunit of the viral glycoprotein.

25. The method of claim 24, wherein the orthologous epitope is situated in the glycan cap region of GP-1.

26. The method of claim 25, wherein the epitope is contained within the consensus sequence I/V/M-G-E-W-A-F-W-E-N/T-K-K-N-F/L-T/S-K/Q/E.

27. The method of claim 26, wherein the epitope comprises the amino acid sequence G-E-W-A-F.

28. The method of claim 24, wherein the orthologous epitope is situated at the tip of the fusion loop of the filovirus glycoprotein.

29. The method of claim 28, wherein the epitope is contained within the consensus sequence A-I/A-G-L/I-A-W-I-P-Y-F-G-P-A/G-A in BDBV, TAFV, RESTV, SUDV, or EBOV, or is contained within the consensus sequence A-A-G-L-S-W-I-P-F-F-G-P-G-I in MARV or RAVN.

30. The method of claim 29, wherein the epitope comprises the amino acid sequence A-I-G-L-A-W-I-P-Y-F, A-A-G-L-A-W-I-P-Y-F, A-A-G-I-A-W-I-P-Y-F, or A-A-G-L-S-W-I-P-F-F.

31. The method of claim 8 or 18, wherein the at least one, at least two, at least three, at least four, or more antibodies are murine antibodies, non-human primate (NHP) antibodies, humanized antibodies, chimeric antibodies, fragments thereof, or any combination thereof.

32. The method of claim 8 or 18, wherein the at least one, at least two, at least three, at least four, or more antibodies are monoclonal antibodies, components of a polyclonal antibody mixture, recombinant antibodies, multispecific antibodies, fragments thereof, or any combination thereof.

33. The method of claim 32, wherein the at least one, at least two, at least three, at least four, or more antibodies are bispecific antibodies or fragments thereof.

34. The method of claim 33, wherein the at least one, at least two, at least three, at least four, or more bispecific antibodies can recognize two different surface exposed and accessible epitopes on a filovirus virion particle.

35. The method of claim 34, two different epitopes can be situated in the glycan cap of the filovirus glycoprotein, the tip of the fusion loop of the filovirus glycoprotein, the mucin-like domain, the GP2 fusion domain, or any combination thereof.

36. The method of claim 8, 18, or 31, wherein the at least one, at least two, at least three, at least four, or more antibodies comprise a heavy chain constant region or fragment thereof.

37. The method of claim 36, wherein the heavy chain constant region or fragment thereof is a murine constant region or fragment thereof, a macaque constant region or fragment thereof, or a human constant region or fragment thereof.

38. The method of claim 36, wherein the heavy chain constant region or fragment thereof is an IgM, IgG, IgA, IgE, IgD, or IgY constant region or fragment thereof.

39. The method of claim 36, wherein the at least one, at least two, at least three, at least four, or more antibodies further comprise a light chain constant region or fragment thereof.

40. The method of claim 39, wherein the light chain constant region or fragment thereof is a murine constant region or fragment thereof, a macaque constant region or fragment thereof, or a human constant region or fragment thereof.

41. The method of claim 8, 18, or 31, wherein the at least one, at least two, at least three, at least four, or more antibodies comprise an Fv fragment, an Fab fragment, an F(ab′)2 fragment, an Fab′ fragment, a dsFv fragment, an scFv fragment, an scFab fragment, an sc(Fv)2 fragment, or any combination thereof.

42. The method of claim 8, 18, or 31, wherein the at least one, at least two, at least three, at least four, or more antibodies are conjugated to an antiviral agent, a protein, a lipid, a detectable label, a polymer, or any combination thereof.

43. The method of claim 1, wherein the filovirus infection is hemorrhagic fever.

44. The method of claim 1, wherein the subject is a nonhuman primate or a human.

45. An isolated, non-naturally-occurring peptide consisting of no more than 25, 50, 75, or 100 amino acids comprising the amino acid sequence G-E-W-A-F.

46. The isolated, non-naturally-occurring peptide of claim 45 comprising the amino acid sequence X1-G-E-W-A-F-W-E-X2-K-K-N-X3-X4-X5, wherein X1 is I, V, or M, X2 is N or T, X3 is F or L, X4 is T or S, and X5 is K, Q, or E.

47. An isolated, non-naturally-occurring peptide consisting of no more than 25, 50, 75, or 100 amino acids comprising the amino acid sequence A-I-G-L-A-W-I-P-Y-F, A-A-G-L-A-W-I-P-Y-F, A-A-G-I-A-W-I-P-Y-F, or A-A-G-L-S-W-I-P-F-F.

48. The isolated, non-naturally-occurring peptide of claim 47, comprising the amino acid sequence A-X1-G-X2-A-W-I-P-Y-F-G-P-X3-A, wherein X1 is I or A, X2 is L or I, and X3 is A or G, or the amino acid sequence A-A-G-L-S-W-I-P-F-F-G-P-G-I.

49. The isolated, non-naturally-occurring peptide of claim 45, which is attached to a heterologous moiety.

50. The isolated, non-naturally-occurring peptide of claim 49, wherein the heterologous moiety comprises an antimicrobial agent, a therapeutic agent, a prodrug, a peptide, a linker, a scaffold, a protein, an enzyme, a lipid, a biological response modifier, a pharmaceutical agent, a lymphokine, a heterologous antibody or fragment thereof, a detectable label, a polyethylene glycol (PEG), and any combination thereof.

51. An immunogenic composition comprising the isolated, non-naturally-occurring peptide of claim 45.

52. A method of producing an antibody or antigen-binding fragment thereof that specifically binds to an orthologous filovirus glycoprotein epitope, comprising administering the immunogenic composition of claim 51 to an animal, and recovering the antibody or fragment thereof.
